(19) United States
(12) Patent Application Publication
(10) Pub. No.: US 2017/0176436 A1
(43) Pub. Date: Jun. 22, 2017

(54) METHODS (AMACR)

(71) Applicant: UNIVERSITY OF BATH, Claverton Down, Bath Bath & North East (GB)

(72) Inventors: MATTHEW LLOYD, BATH, BATH AND N.E. SOMERSET (GB); TIMOTHY WOODMAN, WARMINSTER WILTSHIRE (GB); MAKSIMS JEVGLEVSKIS, BRISTOL AVON (GB); MICHAEL THREADGILL, Batheaston, Bath Bath & N.E. Somerset (GB); GUAT LEE, BARNET HERTFORDSHIRE (GB)

(73) Assignee: UNIVERSITY OF BATH, (GB)

(21) Appl. No.: 15/115,851

(22) PCT Filed: Feb. 2, 2015
(86) PCT No.: PCT/GB2015/050277

§ 371 Date: Aug. 1, 2016

(30) Foreign Application Priority Data
Feb. 3, 2014 (GB) 1401817.0

(51) Int. Cl. G01N 33/574; C07H 19/207; A61K 51/04; A61K 49/04; A61K 49/10; G01N 33/52; A61K 49/00

(52) U.S. Cl. CPC G01N 33/574; G01N 33/52; C07H 19/207; A61K 49/0052; A61K 49/04; A61K 49/10; A61K 51/0491; G01N 2333/99; C07B 2200/05

(57) ABSTRACT

A method of measuring α-methylacyl-CoA racemase (AMACR) activity in a sample is described comprising the steps of: (i) providing a sample; (ii) contacting said sample with a substrate compound that undergoes elimination of hydrogen and a leaving group catalysed by said AMACR to produce a product compound having an additional carbon-carbon double bond; and (iii) measuring the amount of said leaving group and/or of said product compound and/or of unreacted substrate compound. Also provided are methods of diagnosis and medical imaging, in particular for use in diagnosing prostate cancer. Also provided are substrate compounds of Formula 1 for use in the methods described herein.

### FIELD OF THE INVENTION

The present invention relates to methods of measuring the activity of a polypeptide enzyme, α-methylacyl-CoA racemase (AMACR). The invention further relates to compounds for use in such methods, and methods of diagnosis and medical imaging.

### BACKGROUND

α-Methylacyl-CoA racemase (AMACR) is a mitochondrial and peroxisomal enzyme that catalyses the racemization of α-methyl, branched carboxylic coenzyme A thioesters. It is important in the oxidation of bile acid intermediates such as di- and trihydroxycholestanoic acid (DHCA and THCA) and branched-chain fatty acids such as pristanic acid. The enzyme requires no cofactors and catalyzes its reaction by a stepwise 1,1-proton transfer via an enolate intermediate. AMACR is also known as P504S.

The gene encoding AMACR is over-expressed and under-expressed in certain diseases. For example, AMACR is over-produced in all prostate cancers. Over-expression of the AMACR gene is also thought to be a feature of a number of other cancers, including colorectal cancer, breast cancer, ovarian cancer and melanoma.

Quantitative analysis of AMACR therefore offers an attractive additional or alternative route to diagnosis, prognosis and monitoring of disease. Moreover, AMACR inhibitors may have utility in disease treatments, for example, in the treatment of castrate-resistant prostate cancers. Accordingly, the development of a convenient assay of AMACR activity would be highly useful for the development and testing of such inhibitors for use as drugs.

Current methods for measurement of the amount of AMACR polypeptide based on immunoassay suffer from a number of drawbacks common to such methods. For example, they have relatively low accuracy and precision. They measure the total amount of AMACR, not the functional activity of AMACR. There are several different types of AMACR which are believed to have different levels of activity and hence different levels of importance in the disease process. These different types of AMACR cannot at present be distinguished using existing methods. Methods for measuring activity also suffer from been low-throughput, are not easy to utilise with clinical samples, and require specialised equipment to measure radioactivity, circular dichroism or changes in NMR spectra.

The present invention addresses the above-identified problems and further provides additional advantages.

### SUMMARY OF THE INVENTION

The present invention exploits a previously unreported elimination reaction that is catalysed by AMACR. Advantageously, the invention does not require the use of antibodies, and can provide a measurement of the function or activity of AMACR in a sample. Methods for assessing AMACR activity are described which can be used to simultaneously assess many samples. The methods can also allow quantification of results at a much higher level of precision and accuracy than is currently possible. The measured activity of AMACR correlates with the biological activity of AMACR in vivo, and thereby provides a more useful assay than the prior art methods that measure total amount of AMACR protein.

In a first aspect, the present invention provides a method of measuring α-methylacyl-CoA racemase (AMACR) activity in a sample comprising the steps of: (i) providing a sample; (ii) contacting said sample with a substrate compound that undergoes elimination of hydrogen and a leaving group catalysed by said AMACR to produce a product compound having an additional carbon-carbon double bond; and (iii) measuring the amount of said leaving group and/or of said product compound and/or of unreacted substrate compound. In certain embodiments, the measurement may be made by comparison with a reference or a control sample.

Suitably, the hydrogen and the leaving group in the substrate compound are bonded to adjacent carbon atoms, whereby the elimination results in formation of a carbon-carbon double bond between the adjacent carbon atoms. In embodiments, the carbon-carbon double bond is conjugated to one or more unsaturated moieties in the product compound.

Suitably, the hydrogen in the substrate is bonded to a carbon atom further having a hydrogen, methyl or methyl mimetic substituent. The hydrogen, methyl or methyl mimetic substituent binds to a methyl binding pocket of AMACR. Suitably, the leaving hydrogen in the substrate is bonded to a carbon atom further having a carbonyl substituent. The carbonyl substituent forms part of the enolate intermediate of the elimination reaction. Suitably, the carbonyl group forms part of an ester, thioester, amide, or carbonylmethyleneoxy linkage to a CoA residue. The CoA residue promotes binding of the substrate to AMACR.

Suitably, the step of measuring comprises measuring NMR spectra or UV/visible absorption or fluorescence of the leaving group and/or of the product compound and/or of the unreacted substrate compound. Alternatively or additionally, the step of measuring may comprise measuring circular dichroism or optical rotation, since the starting compound has at least one chiral centre that is lost in the elimination reaction.

Suitably, the leaving group is fluorine, which is eliminated as fluoride ions in solution. Concentration of fluoride in solution can be determined by several well-established methods as detailed below.

Suitably, the leaving group is a substituted or unsubstituted phenoxide group. Suitably the substituted or unsubstituted phenoxide group is a substituted or unsubstituted nitrophenoxide group. Suitably the substituted or unsubstituted nitrophenoxide group is a substituted or unsubstituted mono-nitrophenoxide, di-nitrophenoxide or tri-nitrophenoxide group.

Suitably, the substrate compound has formula I:

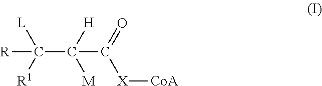

wherein: L is the leaving group; M is a hydrogen, methyl group or a methyl-mimetic group; R¹ is H or an R group as defined below; X is O, S, CH₂, C1-C7 alkyleneoxy, C1-C7 alkylenethio or NH; and R is a group selected from substituted or unsubstituted alkyl, aryl, (hetero)alkyl, (hetero)alkenyl, (hetero)alkynyl, (hetero)aryl, arylalkyl, (hetero)arylalkyl, cycloalkyl, (hetero)cyclyl, cycloalkylaryl, (hetero)cycloalkyl, (hetero)cycloalkylaryl, heterocyclylalkyl, a peptide, an oligosaccharide, a steroid group or H.

Suitably, L is F, or is a substituted or unsubstituted mono-nitrophenoxide, di-nitrophenoxide or tri-nitrophenoxide group;

M is H or methyl; R¹ is H or C1-C7 alkyl, and/or X is S, O, NH, CH₂, CH₂S or CH₂O.

Suitably, L is F, M is H or methyl, R¹ is H or C1-C7 alkyl, and/or X is S, O, NH, CH₂, CH₂S or CH₂O.

Suitably, L is:

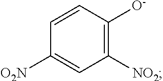 [SMILES: O=[N+]([O-])c1ccc([O-])c([N+](=O)[O-])c1]

M is methyl; R¹ is H or C1-C7 alkyl; R is substituted or unsubstituted alkyl, or H; and/or X is S, O, NH, CH₂, CH₂S or CH₂O.

Suitably, the sample is a biological sample removed from a subject, optionally with further processing steps before testing, or purified AMACR protein, or a recombinant AMACR protein.

In a further aspect, the present invention provides a method for diagnosing and/or detecting a disease in a subject, comprising the steps of: (i) providing a sample from a subject; (ii) measuring the AMACR activity in the sample according to the method described herein; (iii) comparing the measurement from step (ii) with a reference standard; and (iv) using the comparative measurement from step (iii) to determine whether the subject has a disease; (v) optionally, treating said subject if said subject has the disease.

The method is expected to be especially useful for diagnosis of cancer, in particular prostate cancer. The method is also expected to be useful for diagnosis of Anisakisis and AMACR function deficiency.

In a further aspect, the present invention provides a method of monitoring the metastasis of a cancer in a subject, the method comprising the steps: (i) providing samples from a subject obtained at first and second time points; (ii) measuring the AMACR activity in the samples as described herein; and (iii) comparing the at least two measurements from step (ii) with each other; wherein an increase in AMACR activity in the sample taken at the later time point compared to the corresponding level of AMACR activity in the sample taken at the earlier time point is indicative of an increase in the number of circulating cancer cells in the patient, and wherein a decrease in the level of AMACR activity in the sample taken at the later time point compared the corresponding level of AMACR activity in the sample taken at the earlier time point is indicative of a decrease in the number of circulating cancer cells in the patient.

In a further aspect, the present invention provides a method of monitoring the effectiveness of an anti-cancer therapy in a subject, the method comprising the steps: (i) providing samples from a subject obtained at first and second time points, wherein the subject has been treated with an anti-cancer therapy before the two time points or in the interval between the first and second time points, (ii) measuring the AMACR activity in the samples as described herein; and (iii) comparing the at least two measurements from step (ii); wherein a decrease in the level of AMACR activity in the sample taken at the later time point compared the corresponding level of AMACR activity in the sample taken at the earlier time point is indicative of the anti-cancer therapy being efficacious, and wherein an increase in the level of AMACR activity in the sample taken at the later time point compared the corresponding level of AMACR activity in the sample taken at the earlier time point is indicative of the anti-cancer therapy being non-efficacious.

In a further aspect, the present invention provides a method of assaying for the presence of a biomarker which is indicative of cancer cells in a sample, the method comprising the steps: (i) providing a sample from a subject; (ii) measuring the AMACR activity in the sample according to the methods described herein; and (iii) comparing the measurement from step (ii) with a reference standard; wherein an increase in AMACR activity in the biological sample as compared to the reference standard is indicative of cancer cells being present in the biological sample.

In a further aspect, the present invention provides a compound of Formula I as defined above, wherein the compound undergoes elimination of H and the leaving group L catalysed by AMACR to produce a product compound of Formula II:

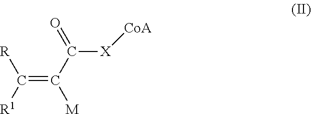

Or an E/Z isomer thereof, wherein R, R¹, M and X are as defined above in relation to Formula (I)

The compounds according to the present invention are suitable for use in the methods of measurement or diagnosis according to the invention.

In a further aspect, the present invention provides a compound according to the invention for use in a method of diagnostic imaging of AMACR activity in a subject, comprising: administering the compound to a subject, followed by imaging the distribution of the leaving group and/or of the product compound and/or of unreacted substrate compound in the subject.

In a further aspect, the present invention provides a method for identifying a compound that modulates the activity of AMACR comprising the steps of: (i) providing a sample comprising AMACR; (ii) providing at least one test compound; (iii) contacting said sample in the presence and absence of said test compound with a substrate compound that undergoes elimination of hydrogen and a leaving group catalysed by said AMACR to produce a product compound having an additional carbon-carbon double bond; and (iv) measuring the amount of said leaving group and/or of said product compound and/or of unreacted substrate compound in the presence and absence of said test compound, wherein a change in the amount of said leaving group and/or of said product compound and/or of unreacted substrate compound in the presence and absence of said test compound is indicative that said test compound modulates the activity of AMACR. The AMACR used in this method may, for example, be recombinant AMACR as described herein. The methods according to this aspect of the invention can be used for development of e.g. AMACR inhibitor drugs.

In a further aspect, the present invention provides a kit for measuring AMACR activity in a sample comprising: (i) a substrate compound that undergoes elimination of hydrogen and a leaving group catalysed by AMACR to produce a product compound having an additional carbon-carbon double bond or the substrate compound described herein; (ii) a positive control that reacts with the substrate compound that is indicative of AMACR activity; and/or (iii) a negative control that does not react with the substrate compound.

In a further aspect, the present invention provides the use of a substrate compound that undergoes elimination of hydrogen and a leaving group catalysed by AMACR to produce a product compound having an additional carbon-carbon double bond or the use of the substrate compound according to claim 16 for measuring AMACR activity in a sample.

In a further aspect, the present invention provides the use of a substrate compound that undergoes elimination of hydrogen and a leaving group catalysed by AMACR to produce a product compound having an additional carbon-carbon double bond or the use of the substrate compound described herein for diagnosing a disease.

In a further aspect, the present invention provides the use of a substrate compound that undergoes elimination of hydrogen and a leaving group catalysed by AMACR to produce a product compound having an additional carbon-carbon double bond or the use of the substrate compound described herein for the diagnostic imaging of AMACR function or activity.

### BRIEF DESCRIPTION OF THE FIGURES

FIG. 1: shows the ¹H NMR spectrum of the aromatic region of the assay sample incubated with active (live) AMACR enzyme as described in Example 5;

FIG. 2: shows the ¹H NMR spectrum of the aromatic region of the negative control incubated with heat-inactive (boiled) AMACR enzyme as described in Example 5;

FIG. 3: shows the Michaelis-Menten plot from the kinetic measurements described in Example 5; and

FIG. 4: shows the Lineweaver-Burk plot from the same kinetic measurements described in Example 5.

### DETAILED DESCRIPTION

The technical terms and expressions used within the scope of this application are generally to be given the meaning commonly applied to them in the pertinent art of chemistry, biology and molecular biology. The word “comprising” does not exclude other elements or steps, and the indefinite article “a” or “an” does not exclude a plurality. A single step may fulfil the functions of several features recited in the claims. The terms “about”, “essentially” and “approximately” in the context of a given numerate value or range refers to a value or range that is within about 20%, within about 10%, or within about 5%, about 4%, about 3%, about 2% or about 1% of the given value or range.

The present disclosure provides a method of measuring AMACR polypeptide activity in a sample. As discussed above, AMACR is a mitochondrial and peroxisomal enzyme that catalyses the racemization of α-methyl, branched carboxylic coenzyme A thioesters. The cDNA sequence of a human gene that encodes the AMACR polypeptide is published in GenBank under the Accession Number NM_014324.5 (see SEQ ID NO:1, which discloses the nucleic acid sequence of the AMACR 1A gene). The term “AMACR polypeptide” refers to a polypeptide that encodes AMACR. The amino acid sequence of a published human AMACR polypeptide is set forth in SEQ ID NO:2. This term includes polypeptide variants that function as AMACR polypeptides—such as polypeptide variants comprising, consisting or consisting essentially of an amino acid sequence encoded by a polynucleotide with at least about 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99% or 100% sequence identity to the polynucleotide sequence encoding human AMACR; polypeptide variants having at least about 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99% or 100% sequence identity to the human AMACR polypeptide; functional fragments of the human AMACR polypeptide; and functional fragments that have at least about 60%, 61%, 62%, 63%, 64%, 65%, 70%, 75%, 80%, 85%, 87%, 88%, 89%, 90%, 91%, 92%, 93%, 94%, 95% 96%, 97%, 98%, 99% or 100% sequence identity to the corresponding fragments of the human AMACR polypeptide. The AMACR polypeptide can also include sequences comprising a sufficient or substantial degree of identity or similarity to the human AMACR polypeptide that function as an AMACR. The fragments of the AMACR polypeptide retain AMACR activity. AMACR polypeptides also include variants and mutants produced by introducing any type of alterations (for example, insertions, deletions, or substitutions of amino acids; changes acids; changes in glycosylation states; changes that affect refolding or isomerizations, three-dimensional structures, or self-association states), which can be deliberately engineered or isolated naturally provided that they still function as an AMACR. AMACR polypeptides may be in linear form or cyclized using known methods. The term also refers to a polypeptide comprising, consisting or consisting essentially of the sequence set forth in GenBank Accession Number ABQ59031 with 100% sequence identity thereto. The term can also encompass splice variants and variants with polymorphisms—such as single nucleotide polymorphisms. Amino acid sequences of examples of published human AMACR variants (eg. splice variants) are set forth in SEQ ID NOs: 3-9. cDNA sequences of examples of AMACR splice variants are set forth in SEQ ID NOs. 12-17.

The term can also encompass isoforms of AMACR that have AMACR activity. Splice variants of human AMACR are described in, for example, Shen-Ong et al., Cancer Res. 2003; 63:3296-301; Mubiru et al., Gene 2004; 327:89-98; Mubiru et al., Prostate 2005; 65:117-23; and Ouyang et al. Urology 2011; 77. 249 e1-e7.

A number of AMACR polymorphisms (for example, single nucleotide polymorphisms)—such as SNPs associated with increased risk of prostate and colon cancer—and are known in the art and are contemplated for use in the present disclosure.

AMACR polypeptides also include variants produced by introducing any type of alterations (for example, insertions, deletions, or substitutions of amino acids; changes in glycosylation states; changes that affect refolding or isomerizations, three-dimensional structures, or self-association states), which can be deliberately engineered or isolated naturally. The variant may have alterations which produce a silent change and result in a functionally equivalent polypeptide. Deliberate amino acid substitutions may be made on the basis of similarity in polarity, charge, solubility, hydrophobicity, hydrophilicity and the amphipathic nature of the residues as long as the function of the polypeptide is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine. Conservative substitutions may be made, for example according to the Table below. Amino acids in the same block in the second column and preferably in the same line in the third column may be substituted for each other:

| | | |
|---|---|---|
| ALIPHATIC | Non-polar | Gly Ala Pro |
| | | Ile Leu Val |
| | Polar - uncharged | Cys Ser Thr Met |
| | | Asn Gly |
| | Polar - charged | Asp Glu |
| | | Lys Arg |
| AROMATIC | | His Phe TrpTyr |

The polypeptide may be a mature polypeptide or an immature polypeptide or a polypeptide derived from an immature polypeptide.

AMACR polypeptides may be or may be derived from any source, natural or synthetic. AMACR polypeptides may be isolated from a known source of the enzyme—such as the cytosol, mitochondria, peroxisomes, microsomes, endoplasmic reticulum and the like. In one example, AMACR polypeptides are isolated from liver or kidney cytosol or mitochondria by one or more known techniques that include centrifugation, dialysis, gel filtration chromatography, ammonium sulfate precipitation, chromatography —such as DEAE-sepharose chromatography, hydroxyl apatite chromatography, phenyl-sepharose CL-4B chromatography, red dye affinity chromatography, or sephacryl S-200 chromatography. See, for example, Shieh, et al., J. Biol. Chem. 1993. 268(5):3487-3493. AMACR may be obtained using recombinant techniques (see, for example, Amery, et al., J. Lipid Res. 2000 41:1752-1759, Kotti, et al., J. Biol. Chem. (2000) 275(27):20887-20895, D. J. Darley et al., Org. Biomol. Chem., 2009, 7, 543-552, and A. J. Carnell, et al., ChemMedChem, 2013, 8, 1643-1647). Other sources of AMACR polypeptides include chemical synthesis of the polypeptide or use of cDNA or a synthetic oligonucleotide encoding the sequence of AMACR to produce the polypeptide in an expression system. Such an expression system may include an insect cell expression system, mammalian cell expression system, yeast cell expression system, bacterial expression system, or plant expression system and the like.

Suitably, the AMACR polypeptide is of mammalian origin, such as human origin. Alternatively, the AMACR (or other polypeptide having AMACR function or activity) may be derived from any other prokaryotic or eukaryotic species.

In embodiments, AMACR activity of AMACR itself, or closely related polypeptides, including closely related polypeptides from any Mycobacterial species is measured, for example from M. tuberculosis. An exemplary published amino acid sequence is set forth in SEQ ID NO: 10. Accordingly, Mycobacterial polypeptides and Mycobacterial polypeptide variants are contemplated that function as AMACR polypeptides—such as polypeptide variants comprising, consisting or consisting essentially of an amino acid sequence encoded by a polynucleotide with at least about 40%, 50%, 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99% or 100% sequence identity to the polynucleotide sequence encoding Mycobacterial AMACR; polypeptide variants having at least about 40%, 50%, 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99% or 100% sequence identity to the Mycobacterial AMACR polypeptide; functional fragments of the Mycobacterial AMACR polypeptide; and functional fragments that have at least about 40%, 50%, 60%, 61%, 62%, 63%, 64%, 65%, 70%, 75%, 80%, 85%, 87%, 88%, 89%, 90%, 91%, 92%, 93%, 94%, 95% 96%, 97%, 98%, 99% or 100% sequence identity to the corresponding fragments of the Mycobacterial AMACR polypeptide. The AMACR polypeptide can also include sequences comprising a sufficient or substantial degree of identity or similarity to the Mycobacterial AMACR polypeptide that function as an AMACR. The fragments of the AMACR polypeptide retain AMACR activity.

In embodiments, the disclosure measures AMACR activity of AMACR itself, or closely related polypeptides from the parasite Anisakis simplex. An exemplary published amino acid sequence is set forth in SEQ NO: 11. Accordingly, Anisakis polypeptides and Anisakis polypeptide variants are contemplated that function as AMACR polypeptides—such as polypeptide variants comprising, consisting or consisting essentially of an amino acid sequence encoded by a polynucleotide with at least about 40%, 50%, 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99% or 100% sequence identity to the polynucleotide sequence encoding Anisakis AMACR; polypeptide variants having at least about 40%, 50%, 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99% or 100% sequence identity to the Anisakis AMACR polypeptide; functional fragments of the Anisakis AMACR polypeptide; and functional fragments that have at least about 40%, 50%, 60%, 61%, 62%, 63%, 64%, 65%, 70%, 75%, 80%, 85%, 87%, 88%, 89%, 90%, 91%, 92%, 93%, 94%, 95% 96%, 97%, 98%, 99% or 100% sequence identity to the corresponding fragments of the 30 Anisakis AMACR polypeptide. The AMACR polypeptide can also include sequences comprising a sufficient or substantial degree of identity or similarity to the Anisakis AMACR polypeptide that function as an AMACR. The fragments of the AMACR polypeptide retain AMACR activity.

In order to determine the percentage identity between two amino acid or polynucleotide or polypeptide sequences, an alignment of the two sequences is performed, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants. Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance. The popular multiple alignment program ClustalW (Thompson et al, 1994, Nucleic Acids Research, 22, 4673-4680; Thompson et al, 1997, Nucleic Acids Research, 24, 4876-4882) is a suitable way for generating multiple alignments. Suitable parameters for ClustalW maybe as follows: For DNA alignments: Gap Open Penalty=15.0, Gap Extension Penalty=6.66, and Matrix=Identity. For protein alignments: Gap Open Penalty=10.0, Gap Extension Penalty=0.2 and Matrix=Gonnet. For DNA and Protein alignments: ENDGAP=−1, and GAPDIST=4. Suitably, calculation of percentage identities between two amino acid or polynucleotide or polypeptide sequences is then calculated from such an alignment as (N/T)*100, where N is the number of positions at which the sequences share an identical residue, and T is the total number of positions compared including gaps but excluding overhangs. Hence, a suitable method for calculating percentage identity between two sequences comprises (i) preparing a sequence alignment using the ClustalW program using a suitable set of parameters, for example, as set out above; and (ii) inserting the values of N and T into the following formula (N/T)*100.

The measured activity of AMACR in the methods described herein correlates with the biological activity of AMACR in vivo, and thereby provides a more useful assay than the prior art methods that measure only total AMACR.

As mentioned above, AMACR in nature catalyses the racemization of alpha-methyl, branched carboxylic coenzyme A thioesters. That is to say, it catalyses the racemization of compounds of formula A:

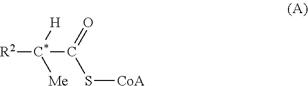

wherein C* indicates the chiral α-carbon atom that undergoes racemization. The Coenzyme A (CoA) group is thought to contribute to binding of the substrate to the enzyme. The methyl group on the α-carbon atom is thought to bind to a methyl group binding pocket of the AMACR. AMACR tolerates a wide range of substrate terminal groups R², corresponding to the aliphatic side chains of various bile acid intermediates, also aromatic rings (ibuprofen and various drugs) and steroid groups.

In one aspect, the present inventors have now found that AMACR advantageously catalyses a novel α,β-elimination reaction in related compounds of Formula B having a leaving group—such as a fluoride leaving group—on the β-carbon atom:

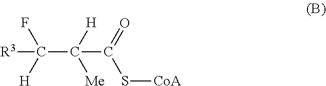

where R³ is an alkyl group, to produce an unsaturated compound of formula (C):

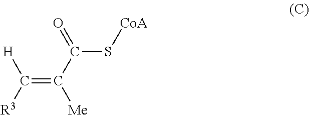

wherein said formula (C) encompasses all E/Z or cis/trans isomers.

The observed rate of this elimination reaction correlates to the biological function or activity of AMACR. This novel elimination reaction provides an assay for the activity of AMACR that has many advantages for clinical or research use.

Suitably, the compounds described herein for use in the present disclosure are synthetic or artificial compounds.

It will be apparent from the foregoing that a very wide range of compounds can be used as the substrate for the methods described herein. For example, other leaving groups besides F can be useful. In addition to the compound of Formula (I), for example, it is expected that conjugated systems such as Formula D could undergo α,δ elimination catalysed by AMACR:

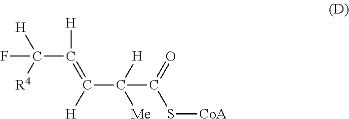

wherein R⁴ is defined in the same way as group R in Formula (I) herein, to produce a conjugated diene of formula (E):

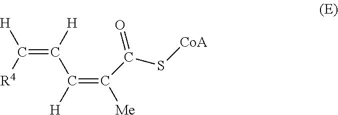

However, suitably, the hydrogen and the leaving group in the substrate compound are bonded to adjacent carbon atoms, whereby the elimination is a simple α,β elimination that results in formation of a carbon-carbon double bond between the adjacent carbon atoms.

Suitably, the hydrogen in the substrate is bonded to a carbon atom further having methyl and carbonyl substituents, preferably methyl and carbonylthio or carboxyl or amide substituents. The methyl substituent contributes to binding of the substrate to AMACR. The carbonyl substituent increases the acidity of the hydrogen and thereby assists the elimination reaction. Suitably, the carbonylthio or carboxyl or amide substituent forms a thioester or ester or amide or carbonyl alkylene oxy linkage to a CoA moiety.

In embodiments, the carbon-carbon double bond formed in the product compound is conjugated to one or more further unsaturated moieties (other than the carbonyl carbon) in the product compound, for example an unsaturated ring in group R. This conjugated system suitably has UV/visible absorption and/or fluorescence maxima in a convenient wavelength range for detection and measurement.

The term “substrate compound” includes substrate compounds that have formula I:

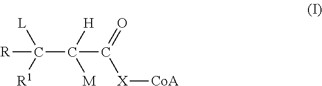

wherein: L is the leaving group; M is hydrogen, a methyl group or a methyl-mimetic group; R¹ is H or an R group as defined below; X is O, S, CH₂, C1-C7 alkyleneoxy, C1-C7 alkylenethio or NH; and R is a group selected from substituted or unsubstituted (hetero)alkyl, (hetero)alkenyl, (hetero)alkynyl, (hetero)aryl, (hetero)arylalkyl, (hetero)cycloalkyl, (hetero)cycloalkylaryl, heterocyclylalkyl, a peptide, an oligosaccharide or a steroid group.

Suitably, the substrate compound has formula I:

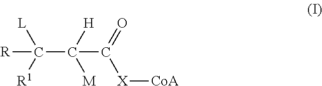

wherein: L is the leaving group; M is a hydrogen, methyl group or a methyl-mimetic group; R¹ is H or an R group as defined below; X is O, S, CH₂, C1-C7 alkylenethio, C1-C7 alkyleneoxy, or NH; and R is a group selected from substituted or unsubstituted alkyl, aryl, heteroalkyl, heteroaryl, arylalkyl, heteroarylalkyl, cycloalkyl, heterocyclyl, cycloalkylaryl, heterocyclylalkyl, heterocyclylalkylaryl, a peptide, an oligosaccharide.

Suitably, the substrate compound is:
(2 S,3R)-3-Fluoro-2-methyldecanoic-CoA

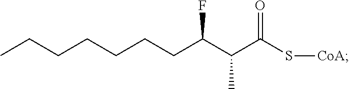

(2R,3S)-3-Fluoro-2-methyldecanoic-CoA

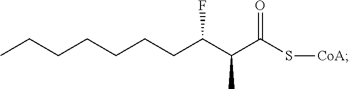

(2R,3R)-3-Fluoro-2-methyldecanoyl-CoA

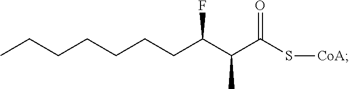

(2S,3S)-3-Fluoro-2-methyldecanoyl-CoA

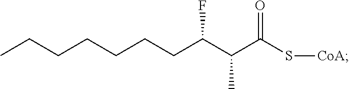

(2R)-3-(2,4-dinitrophenoxy)-2-methyl-propanoyl-CoA

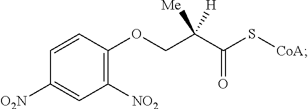

(2S)-3-(2,4-dinitrophenoxy)-2-methyl-propanoyl-CoA

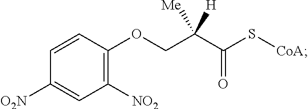

racemic 3-(2,4-dinitrophenoxy)-2-methyl-propanoyl-CoA

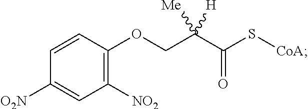

or mixtures thereof.

The term “leaving group” is used in its usual sense in organic chemistry to refer to a molecular fragment that departs with a pair of electrons in heterolytic bond cleavage. Thus, for example, hydrogen and hydrocarbyl groups are not leaving groups. The leaving group may be protonated during the elimination reaction or subsequent to it. Hence, for example, phenoxide anion groups may be protonated to give the corresponding phenol compound, e.g. a mono-nitrophenoxide group may be protonated to give mono-nitrophenol during or subsequent to the elimination reaction. Suitably, the leaving group may be a halide (fluoride, chloride, bromide or iodide), a substituted or an unsubstituted alkyl or aryl sulfonate group, alkyl or aryl sulfonamido group, alkyl or aryl phosphate or phosphonate group, carboxylate group, alkoxide group, phenoxide group, alkyl or aryl thiolate group, alkylthio, arylthio, hydroxyl group, amines, water, alcohols, cyano group or isocyano group.

Suitably, the leaving group is selected from a halide and a substituted or unsubstituted phenoxide group. Suitably, the leaving group is a halide or a substituted or unsubstituted nitrophenoxide group. Substituted or unsubstituted nitrophenoxide groups include substituted or unsubstituted mono-nitrophenoxide, di-nitrophenoxide or tri-nitrophenoxide groups in all isomeric forms. Hence, the substituted or unsubstituted nitophenoxide groups include substituted or unsubstituted 2-nitrophenoxide; 3-nitrophenoxide; 4-nitrophenoxide; 5-nitrophenoxide; 6-nitrophenoxide; 2,3-dinitrophenoxide; 2,4-dinitrophenoxide; 2,5-dinitrophenoxide; 2,6-dinitrophenoxide; 3,4-dinitrophenoxide; 3,5-dinitrophenoxide; 3,6-dinitrophenoxide; 4,5-dinitrophenoxide; 4,6-dinitrophenoxide; 5,6-dinitrophenoxide; 2,3,4-trinitrophenoxide; 2,3,5-trinitrophenoxide; 2,3,6-trinitrophenoxide; 2,4,5-trinitrophenoxide; 2,4,6-trinitrophenoxide; 2,5,6-trinitrophenoxide; 3,4,5-trinitrophenoxide; 3,4,6-trinitrophenoxide; 3,5,6-trinitrophenoxide and 4,5,6-trinitrophenoxide. Suitably the leaving group is a halide or a substituted or unsubstituted mono-nitrophenoxide, di-nitrophenoxide or tri-nitrophenoxide group. Suitably the leaving group is a halide or an unsubstituted mono-nitrophenoxide, di-nitrophenoxide or tri-nitrophenoxide group. Suitably the leaving group is a halide or a substituted or unsubstituted di-nitrophenoxide group. More suitably the leaving group is F, or

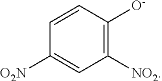 [SMILES: O=[N+]([O-])c1ccc([O-])c([N+](=O)[O-])c1]

The choice of leaving group will depend on factors—such as steric and kinetic factors. Very bulky leaving groups could cause steric hindrance. Very good leaving groups could undergo spontaneous elimination in the absence of AMACR, thereby distorting the assay result. Suitably, the leaving group has intermediate leaving group activity, for example it may be the conjugate base of an acid having a pKa in the range of about −5 to about +6. Suitable leaving groups may include fluoride, chloride and alkyl or aryl carboxylates.

Suitably, the leaving group is fluoride. The use of a fluoride leaving group provides the additional advantage that the product fluoride in solution can be measured readily by standard fluorometric, colourimetric or other methods.

For example, fluoride ion concentration can be measured potentiometrically using a fluoride ion selective electrode (ISE). The fluoride ion selective membrane typically utilizes a membrane consisting of a slice of a single crystal of lanthanum fluoride that has been doped with europium (II) fluoride to improve its conductivity. It is selective to fluoride over other common anions by several orders of magnitude; only hydroxide ion causes serious interference. Fluoride analyses using the ion selective electrode are simple, sensitive, and rapid. A total-ionic strength adjustment buffer (TISAB) is used to adjust samples and standards to the same ionic strength and pH. The pH of the buffer is about 5, a level at which F— is the predominant fluorine-containing species.

Alternatively, fluoride ion concentration can be measured colourimetrically. For example, fluoride ions form stable, colourless complexes with certain multivalent ions, such as (AlF₆)³⁻, (FeF₆)³⁻, and (ZrF₆)³⁻. Most colourimetric methods for the determination of fluoride are based on the bleaching of coloured complexes of these metals with organic dyes when fluoride is added. The degree of bleaching is determined with a spectrophotometer, and the concentration of fluoride ions is assessed by comparison with standard solutions. Common methods for detecting fluoride also include removal of silyl protecting groups from fluorescent or coloured dyes, which allows development of colour/fluorescence. Exemplary methods are described in J. Org. Chem (2011) 76, 3820-3828 and Chem. Eur. J. (2013) 19, 936-942).

A further advantage of fluoride is that it is a relatively poor leaving group, whereby background elimination in the absence of AMACR is reduced.

Suitably the leaving group is

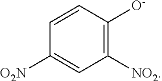 [SMILES: O=[N+]([O-])c1ccc([O-])c([N+](=O)[O-])c1]

Suitably, R is H or optionally substituted C1-C7 alkyl or C1-C7 heteroalkyl. More suitably R is H or C1-C7 alkyl.

Suitably, R₁ is H or optionally substituted C1-C7 alkyl or C1-C7 heteroalkyl. More suitably R₁ is H or C1-C7 alkyl. Most suitably R¹ is H

M is the group that is thought to bond to the α-methyl-binding pocket in AMACR. M may be hydrogen, methyl, or a methyl mimetic group such as trifluoromethyl, trichloromethyl, tribromomethyl, ethyl, or triiodomethyl. Suitably, M is hydrogen, methyl or trifluoromethyl. Most suitably, M is methyl.

X may be any linker group. Suitably, X is O, NH, S, CH₂, C1-C7 alkylenethio or C1-C7 alkyleneoxy, forming with the carbonyl group, respectively, ester, amide, thioester or carbonyl alkylene(oxy) linkages to the CoA group. Suitably, X is O, NH, S, CH₂, CH₂O or CH₂S. Most suitably, X is S.

CoA is the coenzyme A residue or an analogue thereof as described below.

Suitably, the substrate compound comprises a terminal moiety of formula T:

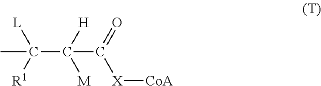

wherein: L is the leaving group as defined above; M is a hydrogen, methyl group or a methyl-mimetic group; R¹ s any R group as defined in relation to Structure 1, suitably H or optionally substituted C1-C7 alkyl or C1-C7 heteroalkyl, most suitably H; X is O, NH, S, CH₂, C1-C7 alkylenethio or C1-C7 alkyleneoxy; and CoA is a coenzyme A residue as defined herein.

The term “coenzyme A” is well known in the biochemistry art. Coenzyme A is present in all living cells that functions as an acyl group carrier and is necessary for fatty acid synthesis and oxidation, pyruvate oxidation, and other acetylation reactions. The following formula shows acetyl CoA having the CoA residue of the formula above bonded to a thioacetyl terminal group:

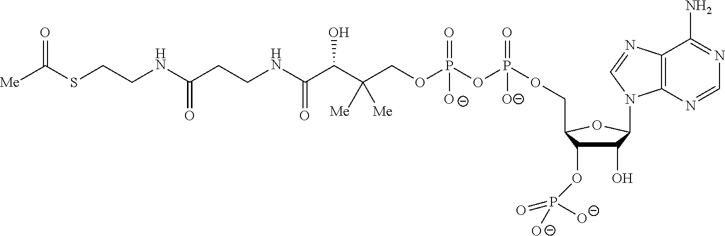

The CoA group is thought to be involved in binding of the substrate compound to AMACR. A number of CoA analogues having similar binding properties are known in the biochemistry art. For example, the terminal sulfide —S— moiety shown in the formula above may be replaced by —CH₂— or —CH₂S—moieties. The term “CoA” herein refers both to the above CoA residue itself, and to any and all such analogues. Most suitably, the CoA group is the above-identified residue of coenzyme A itself.

Most suitably, R¹ is H, M is methyl, and X is S. In these embodiments, the leaving group L is suitably F.

This terminal moiety may be attached to a very wide range of other organic moieties to form a wide range of possible substrate molecules.

In embodiments, the substrate compound has formula I:

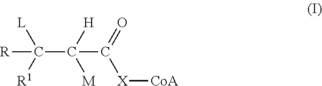

wherein R¹, L, M, X and CoA are as defined above in relation to the terminal moiety of Formula T. R is a group selected from substituted or unsubstituted (hetero)alkyl, (hetero)alkenyl, (hetero)alkynyl, (hetero)aryl, (hetero)arylalkyl, (hetero)cycloalkyl, (hetero)cycloalkylaryl, heterocyclylalkyl, a peptide, an oligosaccharide or a steroid group

Unless otherwise stated, reference in the present specification to an alkyl group includes a branched or unbranched saturated hydrocarbyl radical. Suitably, the alkyl group comprises from about 3 to about 30 carbon atoms, for example from about 5 to about 25 carbon atoms. The term “C₁₋₇ alkyl” refers to straight chain and branched saturated hydrocarbon groups, comprising from 1 to 7 carbon atoms. Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, n-heptyl, and the like.

The term “alkenyl” refers to a branched or unbranched hydrocarbyl radical containing one or more carbon-carbon double bonds. Suitably, the alkenyl group comprises from about 3 to about 30 carbon atoms, for example from about 5 to about carbon atoms.

The term “alkynyl” refers to a branched or unbranched hydrocarbyl radical containing one or more carbon-carbon triple bonds. Suitably, the alkynyl group comprises from about 3 to about 30 carbon atoms, for example from about 5 to about carbon atoms.

R may comprise polycyclic groups including a group comprising two or more non-aromatic carbocyclic or heterocyclic rings which may themselves be substituted.

Reference in the present specification to halogen includes a fluorine, chlorine, bromine or iodine radical, preferably fluorine or chlorine radical.

The term “cycloalkyl” as used herein includes reference to an alicyclic moiety, suitably having 3, 4, 5, 6, 7 or 8 carbon atoms. The group may be a bridged or polycyclic ring system. More often cycloalkyl groups are monocyclic. This term includes reference to groups such as cyclopropyl, cyclobutyl, cyclopentyl, cyclohexyl, norbornyl, bicyclo[2.2.2]octyl and the like.

The term “aryl” as used herein includes reference to an aromatic ring system comprising 6, 7, 8, 9, 10, 11, 12, 13, 14, 15 or 16 ring carbon atoms. Aryl is often phenyl but may be a polycyclic ring system, having two or more rings, at least one of which is aromatic. This term includes reference to groups such as phenyl, naphthyl, fluorenyl, azulenyl, indenyl, anthryl and the like.

The prefix (hetero) herein signifies that one or more of the carbon atoms of the group may be substituted by nitrogen, oxygen, phosphorus, silicon or sulfur.

Heteroalkyl groups include for example, alkyloxy groups and alkythio groups.

Heterocycloalkyl or heteroaryl groups herein may have from 3, 4, 5, 6, 7, 8, 9, 10, 11, 12, 13, 14, 15 or 16 ring atoms, at least one of which is selected from nitrogen, oxygen, phosphorus, silicon and sulfur. In particular, a 3- to 10-membered ring or ring system and more particularly a 5- or 6-membered ring, which may be saturated or unsaturated. For example, selected from oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, especially thiomorpholino, indolizinyl, 1,3-Dioxo-1,3-dihydro-isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, di benzothiophenyl, phthalazinyl, naphthyridinyl, quinoxalyl, quinazolinyl, quinazolinyl, cinnolinyl, pteridinyl, carbazolyl, [beta]-carbolinyl, phenanthridinyl, acridinyl, perimidinyl, phenanthrolinyl, furazanyl, phenazinyl, phenothiazinyl, phenoxazinyl, chromenyl, isochromanyl, chromanyl, 3,4-dihydro-2H-isoquinolin-1-one, 3,4-dihydro-2H-isoquinolinyl, and the like.

Where R is a peptide, the peptide suitably comprises from 1 to 100 amino acid residues, for example from about 2 to about 30 amino acid residues.

Where R is an oligosaccharide, the oligosaccharide suitably comprises from 1 to 100 saccharide residues, for example from about 2 to about 30 saccharide residues.

The term “substituted” as used herein in reference to a moiety includes that one or more, especially up to 5, more especially 1, 2 or 3, of the hydrogen atoms in said moiety are replaced independently of each other by the corresponding number of substituents. The term “optionally substituted” as used herein includes substituted or unsubstituted. It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognised by the skilled person.

Substituents may suitably include halogen atoms and halomethyl groups such as CF₃ and CCl₃; oxygen containing groups such as oxo, hydroxy, carboxy, carboxyalkyl, alkoxy, alkoyl, alkoyloxy, aryloxy, aryloyl and aryloyloxy; nitrogen containing groups such as amino, alkylamino, dialkylamino, cyano, azide and nitro; sulfur containing groups such as thiol, alkylthiol, sulfonyl and sulfoxide; heterocyclic groups which may themselves be substituted; alkyl groups, which may themselves be substituted; and aryl groups, which may themselves be substituted, such as phenyl and substituted phenyl. Alkyl includes substituted and unsubstituted benzyl.

Where two or more moieties are described as being “each independently” selected from a list of atoms or groups, this means that the moieties may be the same or different. The identity of each moiety is therefore independent of the identities of the one or more other moieties.

Suitably, R is a hydrophobic group for binding to a hydrophobic region of AMACR. For example, R is suitably a hydrocarbyl group. Most suitably, R is C3 to C24 alkyl, for example C5 to C16 alkyl, and in certain embodiments it is heptyl. In these embodiments, suitably, R¹ is H, M is methyl, and X is S. Further in these embodiments, the leaving group L is suitably F. In other embodiments, the R group is selected from alkyl groups as hereinbefore defined, optionally substituted aryl groups including phenoxyphenyl, and steroid groups.

Suitably, the substrate compounds for use in the disclosure are soluble in water at 25° C. in an amount of at least 1 mg/mL, more suitably at least about 10 mg/mL.

It will be appreciated that the substrate compounds described herein comprise at least two chiral carbon atoms and may therefore exist as a number of possible stereoisomers and diastereomers. The present inventors have found that the novel elimination reaction does not appear to require the use of any particular stereoisomer or diastereomer of the substrate material. The elimination reaction has been observed in more than one stereoisomer or diastereomer of the same compound. Therefore, the use of all stereoisomers and diastereomers of the above-identified substrate compounds, including mixtures of said isomers, is encompassed in the present disclosure. Suitably, the substrate has an anti-configuration with respect to the methyl (M) and leaving (L) groups, since compounds with a syn-configuration have a greater tendency to eliminate HL spontaneously. The assay reaction in the methods of the present disclosure is suitably carried out in aqueous solution, suitably at a temperature of from about 0° C. to about 50° C., more suitably about 10° C. to about 40° C., typically at about 15° C. to about 37° C. The concentration of the substrate compound in the solution is suitably from about 2×10⁻⁶ mg/mL to about 5 mg/mL, for example from about 2×10⁻⁴ mg/mL to about 1 mg/mL.

As described herein, the methods comprise quantifying the reaction products (and/or of the unreacted starting substrate) of the novel elimination reaction in order to assess the activity of AMACR in the test sample. Suitably, the quantity (concentration) of reaction products is assessed at predetermined time or times after the start of the reaction in order to determine reaction rate information. For example, the quantifying may be performed at one or more intervals of 1, 5, 10, 20, 30 or 60 minutes after the start of the reaction. The reaction mixture may be quenched, for example by heating to inactivate the AMACR e.g. by heating to a temperature of about 50° C. or higher, or by adding acid, base, or organic solvent, prior to carrying out the step of quantifying. Alternatively or additionally, the AMACR may be inactivated by adding one or more protein denaturing agents, such as urea, guanidinium hydrochloride, sodium dodecylsulfate, and other detergents as stop solutions.

The step of quantifying one or more of the reaction products and/or the unreacted substrate may be performed by any analytical method. Suitably, a spectroscopic method is used since such methods are inexpensive and lend themselves to high-throughput operation. Suitable spectroscopic methods include NMR, infrared spectroscopy, luminescence, UV/visible absorption spectroscopy and UV/visible fluorescence spectroscopy. Suitably, the method may be calibrated by reference to standard AMACR solutions of known activity.

Suitably, the step of measuring comprises measuring UV/visible absorption or fluorescence of the leaving group and/or of the product compound and/or of the unreacted substrate compound. Such measurement methods are sensitive, straightforward, low cost, and lend themselves to high-throughput measurement since they do not require further manipulation of the reaction mixture. Furthermore, such methods can be performed continuously, or repeatedly at time intervals, to follow the progress of the reaction and derive rate information.

It is envisaged that the assay reaction in the methods described herein will suitably be performed with the substrate compound present in large excess, whereby the kinetics of the reaction will be substantially first-order with respect to the AMACR activity. That is to say, the rate of evolution of the product compound and the leaving group will be substantially constant for the duration of the assay. Under these conditions, the AMACR activity will be directly proportional to the rate of evolution of the products and can thus be determined simply by comparing the rate of evolution of the products, or the total amount of products evolved after a specific reaction time, with the reaction rates obtained from a standard AMACR sample.

It is envisaged that the present disclosure will have many applications in the fields of medical research, diagnosis and therapy. For example the disclosure could be used to evaluate new drugs which change the levels of AMACR activity, in particular inhibitors which reduce AMACR activity. Such inhibitor drugs could lead to novel treatments for diseases—such as prostate cancer and other cancers. The present disclosure could also be used to evaluate AMACR activity in biological systems in pharmacological and biochemical experiments, thereby helping to elucidate the exact role of AMACR in diseases—such as prostate cancer and other cancers. A particularly promising application of the present disclosure will be for diagnostic measurement of AMACR levels in patients for the detection or monitoring of disease states such as prostate cancer. In these embodiments, the test sample can be a biological sample removed from a subject.

As used herein, the term “subject” refers to any animal, including, but not limited to, mammals, humans, non-human primates, rodents, and the like.

The cancer may be benign or malignant. The cancer may be in the form of circulating cells, e.g. circulating metastatic or metastasizing cancer cells.

There is also provided a method for diagnosing or detecting a disease in a subject, including measuring the activity of AMACR as described herein in a biological sample removed from the subject.

The term “diagnosing” encompasses both detection and identification of a medical condition and can include monitoring the progress of the said condition, for example following treatment. The methods of diagnosis therefore may involve taking a plurality of biological samples at intervals of time to monitor the progress of a medical condition. For example, the plurality of samples may be taken at intervals of from about 1 hour to about 1 year, suitably at intervals of from about 1 day to about 30 days.

The biological sample may suitably be sourced from a specimen of any body tissue or excretion or exudate. Samples include biological fluids—such as blood, plasma, serum and the like—; organ or tissue or cell culture derived fluids; and fluids extracted from physiological tissues. Also included in the term are derivatives and fractions of such fluids. The cells may be dissociated, in the case of solid tissues, or tissue sections may be analysed. Alternatively, a lysate of the cells may be prepared for the purpose of a screening assay. Cell homogenates, differential centrifugation, cellular fractions/extracts, differentiation through the use of protease inhibitors, immunoblotting and enzyme assays and the like may also be employed. The sample may be subjected to further conventional preparation processes, in particular to separation or stabilization processes such as maceration, filtration, ultrafiltration, centrifugation, buffering, dilution, etc. The sample may be of or may be derived from lymph nodes or lymph fluid.

The sample can be compared with a reference standard from another subject. The reference standard may be established by prospective and/or retrospective statistical studies. Healthy subjects who have no clinically evident disease or abnormalities may be selected for statistical studies. Diagnosis may be made by the finding of statistically significant different levels of AMACR function or activity compared to the reference standard.

If the function or activity of AMACR is higher than that of the reference standard, the function or activity of AMACR may be considered to be increased. If the function or activity of AMACR is lower than that of the reference standard, the function or activity of AMACR may be considered to be decreased. If the function or activity of AMACR is higher or lower than that of the reference standard then the function or activity of AMACR in the sample will be considered to be abnormal. If the function or activity of AMACR is increased as compared to the reference standard then this sample is indicative of the presence of disease—such as cancer. If the function or activity of AMACR is decreased as compared to the reference standard then this sample is indicative of functional AMACR deficiency—which can also cause disease. Suitably the reference standard is age matched to the subject. Suitably the reference standard is ethnicity matched to the subject. The reference standard need not necessarily involve the parallel testing of another sample alongside the testing of the sample from the subject of interest. The reference standard may in fact be a numerical value determined on a past occasion. Therefore the comparison with the reference standard may be a purely numerical exercise of comparing the two determined values. When testing samples from the same subject, the samples may be obtained repeatedly, such as daily, every two or three days, weekly, fortnightly or at longer intervals.

Suitably, the change in the level of AMACR activity is a significant change.

In certain embodiments, a significant increase is one where the level of AMACR activity in the sample obtained from the subject is more than 10%, 20%, 30%, 40%, 50%, 60%, 70%, 80%, 90%, 100%, 200%, 300%, 400%, 500%, 600%, 700%, 800%, 900% or 1000% higher as compared to the corresponding level in the sample obtained from the reference standard. In certain embodiments, a significant increase means that the increase is significant using the criteria p<0.05, 2-tailed test.

In certain embodiments, a significant decrease is one where the level of AMACR activity in the sample obtained from the subject is more than a 10%, 20%, 30%, 40%, 50%, 60%, 70%, 80%, 90% or 100% decrease as compared to the corresponding level in the sample obtained from the reference standard. In other embodiments of the invention, a significant decrease means that the decrease is significant using the criteria p<0.05, 2-tailed test.

The methods described herein are expected to be especially useful for detecting or diagnosing a disease related to AMACR activity—such as abnormal or irregular AMACR activity—in a subject. This includes a disease state for which an association with the function or activity of AMACR has been made. For example, AMACR has been associated with, for example, prostate cancer, colon cancer, breast cancer, ovarian cancer, colorectal cancer, bladder cancer, lung cancer, renal cancer, lymphoma and melanoma. In this regard, reference can be made to Cancer Res 2000; 60:1677-82; J Urol 2002; 168:374; Am J Surg Path 2001; 25:1397-404; Am J Clin Pathol 2004; 122:275-89; Cancer Res 2002; 62:2220-6; Human Pathol 2003; 34:792-6; and Am J Pathol 2004; 164:787-93. Other disease states include, by way of illustration and not limitation, inflammation, cystic fibrosis, dementia, neoplastic disease, pain, and so forth. Dementia includes Alzheimer's disease, and so forth. Neoplastic disease includes cancers such as, for example, those cancers mentioned above, and cervical cancer, endometrial cancer, testicular cancer, pancreatic cancer, leukaemia, squamous cell carcinoma, lipoma, brain tumours, and the like. In the diagnosis of prostate cancer, the biological sample is suitably obtained from prostate tissue, semen, or urine.

The method is also expected to be useful for diagnosis of Anisakis by measurement of AMACR function of polypeptides expressed by the parasite Anisakis simplex (see, for example, Korean J Parasitol. 2012 June; 50(2): 165-171).

Suitably, the method further comprises a reference measurement to enable appropriate corrections to normalise the AMACR activity in the biological sample to compensate for variations in dilution, etc. For example, the method may further comprise measuring the total polypeptide content of the test sample and normalising the measured level of AMACR activity to the total polypeptide content. This allows variation in the composition of the test sample to be corrected. For example, total polypeptide content can be determined using the Bradford polypeptide assay (Bradford M M, Anal. Biochem. 1976, 72:248-254).

In a further aspect, the present disclosure provides a compound of Formula I as defined above, wherein the compound undergoes elimination of H and the leaving group L catalysed by AMACR to produce a product compound of Formula II:

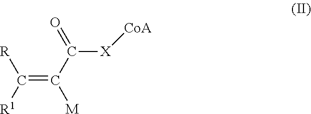

or an E/Z isomer thereof, wherein R, R¹, M and X are as defined above in relation to Formula (I).

The compounds according to the present disclosure are suitable for use in the methods of measurement or diagnosis and the like as described herein.

In a further aspect, the present disclosure provides a compound according to the disclosure for use in a method of diagnostic imaging of AMACR activity in a subject, comprising: administering the compound to a subject, followed by imaging the distribution of the leaving group and/or of the product compound and/or of unreacted substrate compound in the subject. For example, the substrate compounds described above having fluoride leaving groups would lend themselves to such imaging using ¹⁸F for positron emission tomography (PET), or ¹⁹F for NMR and the like.

There is also disclosed a method for imaging a disease associated with AMACR activity comprising performing said imaging with the leaving group and/or of the product compound and/or of the unreacted substrate compound.

There is further described a method for imaging or diagnosing a disease associated with AMACR activity comprising the steps of: administering to a mammal an effective amount of the compound described herein, imaging the distribution of the leaving group and/or of the product compound and/or of unreacted substrate compound; and assessing the images.

In some embodiments, in vivo imaging techniques can be used to visualise AMACR function or activity. Examples of in vivo imaging methods include radionuclide imaging, positron emission tomography (PET), single photon emission tomography (SPECT), computerized axial tomography, X-ray or magnetic resonance imaging method, fluorescence detection, and chemiluminescent detection. The in vivo imaging methods are useful in the diagnosis of diseases as described herein. Such techniques can allow for diagnosis without the use of a biopsy or surgical intervention. The in vivo imaging methods can be useful for providing prognoses to patients. For example, in vivo imaging methods can be used to detect metastatic cancers. Imaging with the leaving group and/or of the product compound and/or of the unreacted substrate compound as described herein can be performed.

Single photon emission tomography (SPECT) and positron emission tomography (PET) may be of particular use as they are compatible with imaging with the leaving group and/or of the product compound and/or of the unreacted substrate compound as described herein. SPECT and PET systems both rely on detection of gamma-ray photons resulting from decay of radio-isotopes, the concentration of which in tissues can be indicative of disease within tissues. For example, some tumours can be characterised by increased AMACR activity and therefore high uptake of the tracer compound, and correspondingly, higher gamma photon emission, while normal tissue exhibits relatively lower AMACR activity and therefore lower uptake of the tracer compound and lower photon emission.

In PET imaging, as a radioactive isotope of the tracer undergoes decay, it emits a positron, an antiparticle of the electron with opposite charge. The emitted positron travels in tissue for a short distance (typically about 1 mm), losing energy as it travels, to a point where it can interact with an electron. The interaction annihilates both the electron and the positron, producing a pair of gamma ray photons having energies of (typically) 511 KeV that move in approximately opposite directions. These high energy photons are detected as a “paired event” (often referred to as “coincidence”) by an opposed pair of detectors. Signals from the detectors are collected and temporally correlated to find such pairs and used to generate or reconstruct the 3D images.

For SPECT imaging, radioactive decay of the tracer isotope results in emission of single gamma-ray photons without the intermediate step of positron-electron annihilation, These photons typically have energies in the range of about 40-245 KeV, and are detected as singular events.

Isotopes used in PET imaging typically have short half-lives. Examples include carbon-11 (approximately 20 min half-life), nitrogen-13 (approximately 10 min), oxygen-15 (approximately 2 min), fluorine-18 (approximately 110 min), or rubidum-82 (approximately 1.27 min). Typical tracers used in SPECT imaging include technetium-99 (the most commonly used), iodine-123, or iodine 131, or indium-111. These isotopes are heavier than those used in PET and exhibit half-lives measured in hours or even days.

PET detector technology is described in Lewellen, Recent Developments in PET Detector Technology, Physics in Medicine and Biology, 53(2008) R-287-317, 11 Aug. 2008. Both conventional PET and SPECT systems are widely used in the fields of oncology and neurology, while SPECT systems are commonly used in cardiology, bone scan imagining, and pre-clinical studies.

A further aspect relates to a method for identifying a compound that modulates the function or activity of AMACR comprising the steps of: (i) providing a sample comprising AMACR; (ii) providing at least one test compound; (iii) contacting said sample in the presence and absence of said test compound with a substrate compound that undergoes elimination of hydrogen and a leaving group catalysed by said AMACR to produce a product compound having an additional carbon-carbon double bond; and (iii) measuring the amount of said leaving group and/or of said product compound and/or of unreacted substrate compound in the presence and absence of said test compound, wherein a change in the amount of said leaving group and/or of said product compound and/or of unreacted substrate compound in the presence and absence of said test compound is indicative that said test compound modulates the activity of AMACR.

A test compound that is subjected to screening may be any compound of interest and includes small organic compounds, polypeptides, peptides, higher molecular weight carbohydrates, polynucleotides, fatty acids and lipids, and the like. Test compounds may be screened individually or in sets or combinatorial libraries of compounds. Test compounds can be obtained from a wide variety of sources including libraries of synthetic or natural compounds. Libraries of natural compounds in the form of bacterial, fungal, plant and animal extracts can be used. Natural or synthetically produced libraries and compounds that are modified through conventional chemical, physical and biochemical means may be used to produce combinatorial libraries. Known pharmacological agents may be subjected to directed or random chemical modifications, such as acylation, alkylation, esterification, acidification to produce structural analogues for screening.

When screening using a combinatorial library, a large library of chemically similar or diverse molecules can be screened. In one approach combinatorial synthesis is employed to prepare a diverse set of molecules in which several components predicted to be associated with the biological activity of AMACR are systematically varied. In combinatorial screening, the number of hits discovered is proportional to the number of molecules tested. The large numbers of compounds, which may reach thousands of compounds tested per day, can be screened using a suitable high throughput screening technique, in which laboratory automation and robotics may be applied. Examples of methods for the synthesis of molecular libraries can be found in the art, for example in: Proc. Natl. Acad. Sci. U.S.A. 90:6909 (1993); Proc. Natl. Acad. Sci. USA 91:11422 (1994); J. Med. Chem. 37:2678 (1994); Science 261:1303 (1993); Angew. Chem. Int. Ed. Engl. 33.2059 (1994); Angew. Chem. Int. Ed. Engl. 33:2061 (1994); and J. Med. Chem. 37:1233 (1994). Libraries of compounds may be presented in solution (see, for example, Biotechniques 13:412-421 (1992)), or on beads (Nature 354:82-84 (1991)), chips (Nature 364:555-556 (1993)), bacteria or spores (U.S. Pat. No. 5,223,409), plasmids (Proc. Natl. Acad. Sci. USA 89:18651869 (1992)) or on phage (Science 249:386-390 (1990); Science 249:404-406 (1990); Proc. Natl. Acad. Sci. 87:6378-6382 (1990); J. Mol. Biol. 222:301 (1991)).

A small organic compound includes a compound of molecular weight less than about 5000, usually less than about 2500, usually, less than about 2000, more usually, less than about 1500, preferably about 100 to about 1000. The small organic compounds may be either biological or synthetic organic compounds. The atoms present in the small organic compound are generally in the group comprising carbon, hydrogen, oxygen, and nitrogen and may include halogens, boron, phosphorus, selenium and sulfur if in a pharmaceutically acceptable form. Generally, oxygen, nitrogen, sulfur or phosphorus, if present, are bound to carbon or one or more of each other or to hydrogen to form various functional groups such as, for example, carboxylic acids, alcohols, thiols, carboxamides, carbamates, carboxylic acid esters, amides, ethers, thioethers, thioesters, phosphates, phosphonates, olefins, ketones, amines, aldehydes, and the like. The small organic compounds, as the term is used herein, also include small peptides, small oligonucleotides, small polysaccharides, fatty acids, lipids, and the like having a molecular weight less than about 5000.

Polypeptides that have a molecular weight of at least about 5,000, more usually at least about 10,000 can be screened. The test polypeptides will generally be from about 5,000 to about 5,000,000 or more molecular weight, more usually from about 20,000 to about 1,000,000 molecular weight. A wide variety of polypeptides may be considered such as a family of polypeptides having similar structural features, polypeptides having particular biological functions, polypeptides related to specific microorganisms, particularly disease causing microorganisms. Such polypeptides include cytokines or interleukins, enzymes, protamines, histones, albumins, immunoglobulins, scleropolypeptides, phosphopolypeptides, mucopolypeptides, chromopolypeptides, lipopolypeptides, nucleopolypeptides, glycopolypeptides, T-cell receptors, proteoglycans, somatotropin, prolactin, insulin, pepsin, polypeptides found in human plasma, blood clotting factors, blood typing factors, polypeptide hormones, cancer antigens, tissue specific antigens, peptide hormones, nutritional markers, tissue specific antigens, and synthetic peptides, which may or may not be glycated.

Polynucleotides can be screened. The test polynucleotide may be a natural compound or a synthetic compound. Polynucleotides include oligonucleotides and are comprised of natural nucleotides such as ribonucleotides and deoxyribonucleotides and their derivatives although unnatural nucleotide mimetics such as 2′-modified nucleosides, peptide nucleic acids and oligomeric nucleoside phosphonates are also contemplated. The higher molecular weight polynucleotides can have from about 20 to about 5,000,000 or more nucleotides.

In another approach an antibody is employed that is capable of binding to AMACR. The antibody is combined with the assay medium and a determination is made as to the level of binding of the antibody to the AMACR. Antibodies may include a complete immunoglobulin or fragment thereof, which immunoglobulins include the various classes and isotypes, such as IgA, IgD, IgE, IgG1, IgG2a, IgG2b, and IgG3, IgM, etc. Fragments thereof may include Fab, Fv and F(ab′)2 Gab′, and the like. In addition, aggregates, polymers, and conjugates of immunoglobulins or their fragments can be used where appropriate so long as binding affinity for a particular molecule is maintained.

Interactions with AMACR include modulating the function or activity of AMACR in living systems—such as animals, plants, animal or plant tissue or cells, transgenic cell lines, transgenic animals, chemically or genetically altered tissue or cells, and so forth. The test compound may influence the activity of AMACR in various ways.

The assay medium used will normally be an aqueous medium. The pH for the medium is usually in the range of about 4.5 to 9.5, more usually in the range of about 5.5-8.5, and preferably in the range of about 6-8. The pH and the temperature will be chosen and varied as the case may be to maximize the ability of the test compound to be screened to influence the activity of AMACR. In addition to buffers, the medium can contain other agents—such as stabilizers, antibiotics, protease inhibitors, nuclease inhibitors, anti-microbial agents, anti-fungal agents, thioesterase inhibitors, pH indicators, solubilizing agents such as detergents and cyclodextrins, proteins such as BSA, solvents such as DMSO, and the like. Moderate temperatures will normally be employed. In general the temperature should be that which is optimal for AMACR activity. The temperature should not be so great as to be detrimental to the reagents used in the assay. The time period for the incubation is generally long enough to permit interaction of AMACR with a test compound and to permit the test compound to influence this interaction if such compound is able to do so. Generally, the time period for incubation is about 1 to about 60 minutes, usually, about 2 to about 30 minutes, and preferably, about 5 to about 10 minutes. The amount employed of the compound to be screened and of AMACR may vary depending on the type of assay, the nature of the compound screened, the activity of AMACR, the purity of the AMACR, the nature (composition) of the incubation medium the source of AMACR and so forth. The screening methods may be designed in a number of different ways, where a variety of assay configurations and protocols may be employed, as are known in the art. All of the reagents may be in solution phase or one of the reagents may be bound to a solid support and the remaining reagents contacted with the support bound reagent. After the appropriate incubation period, the reaction can be terminated by addition of a terminating agent. The extent that the test compound has influenced the activity of AMACR is determined. To this end, one may determine the amount or activity of AMACR, the production, or inhibition of production and so forth.

Kits for conducting any of the above methods can be employed. In one aspect, a kit for measuring AMACR activity in a sample is provided comprising: (i) a substrate compound that undergoes elimination of hydrogen and a leaving group catalysed by AMACR to produce a product compound having an additional carbon-carbon double bond or the substrate compound described herein; (ii) optionally a positive control that reacts with the substrate compound that is indicative of AMACR activity; and/or (iii) optionally a negative control that does not react with the substrate compound. A set of instructions for using the kit to measure AMACR activity may be included. In the kit reagents can be provided in packaged combination in the same or separate containers, depending on the cross-reactivity and stability of the reagents. Other reagents in the kit can include ancillary agents such as buffering agents, stabilizing agents, and the like, calibrators, positive and negative control reagents, salts and so forth. The kit can include supports or surfaces—such as plates having wells—for conducting methods in accordance with the present disclosure. The supports or surfaces may be free of reagents or may contain one or more reagents bound thereto.

Specific embodiments of the invention will now be described further with reference to the following non-limiting examples and to the accompanying drawings.

### Examples

General:

All reactions that require anhydrous conditions were performed under argon atmosphere. Anhydrous and general grade solvents were purchased from the Sigma-Aldrich Chemical Co. and used without further purification. Solvents were removed using Buchi rotary evaporators. Unless otherwise mentioned reactants and reagents were purchased from the Sigma-Aldrich Chemical Co. or Fisher Scientific Ltd. Thin layer chromatography was performed on Merck silica aluminium plates 60 (F254) and UV light, potassium permanganate or phosphomolybdic acid were used for visualization. Water for aqueous solutions was obtained from a Nanopure Diamond system. Column chromatography was performed using Fisher silica gel (particle size 35-70 micron). Purifications of acyl-CoA esters were performed by solid phase extraction using Oasis HLB 6cc (200 mg) extraction cartridges. Phosphate buffer was prepared from monobasic and dibasic potassium phosphates at the required proportion for 0.1 M pH 7.0 buffer. Optical rotations were recorded on Optical Activity AA-10 Automatic polarimeter instrument. IR spectra were recorded on Perkin-Elmer RXI FTIR spectrometer instrument. NMR spectra were recorded on Varian Mercury VX 400 MHz spectrometer in D₂O or CDCl₃ and solvent was used as an internal standard. Shifts are given in ppm and J values reported to 0.1 Hz. Mass spectra were recorded on ESI-TOF at the University of Bath Mass Spectrometry Service.

The following abbreviations are used throughout the specification:

Ac acetyl;

CDI 1,1′-carbonyldiimidazole;

CoA coenzyme A residue;

DAST Diethylaminosulfur trifluoride;

DCM dichloromethane;

Et ethyl;

Me methyl;

OTf trifluoromethanesulfonate;

i-Pr iso-propyl; and

THF tetrahydrofuran.

### Example 1: Synthesis of syn- and anti-3-fluoro-2-methyldecanoyl-CoA esters

The general reaction scheme was as follows:

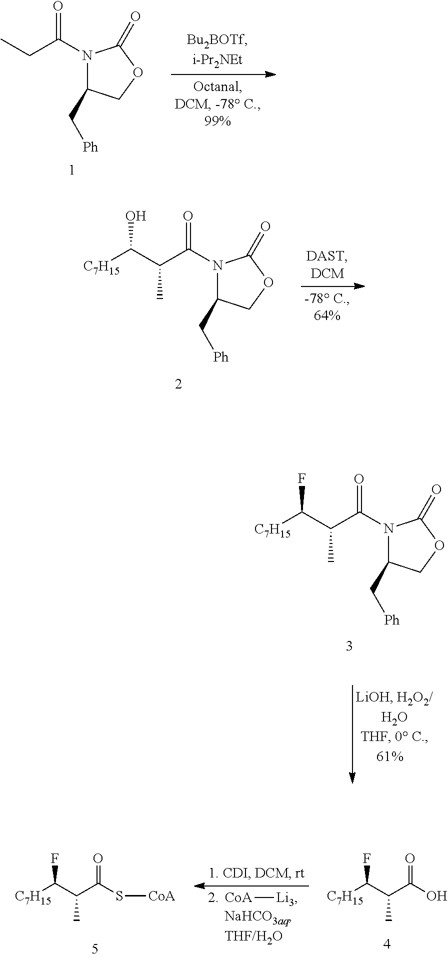

### (S)-4-Benzyl-3-[(2S,3R)-3-hydroxy-2-methyldecanoyl]oxazolidin-2-one (2)

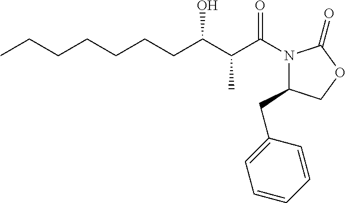 [SMILES: CCCCCCC[C@H](O)[C@@H](C)C(=O)N1C(=O)OC[C@H]1Cc1ccccc1]

A solution of dibutylboron triflate in DCM (1.0 M, 1.30 mL, 1.29 mmol) and diisopropylethylamine (0.25 mL, 1.29 mmol) were added to a stirred solution of oxazolidinone 1 (300 mg, 2.14 mmol) in 10 mL of DCM cooled to −78° C. and the resulting solution was stirred for 30 min. Octanal (0.15 mL, 0.92 mmol) in 3 mL of DCM was added dropwise and the reaction mixture was stirred at −78° C. for 30 min and then allowed to reach ambient temperature. Reaction was quenched by slow addition of phosphate buffer (0.1 M, pH=7, 10 mL), organic layer was then washed with 1M HCl_{aq}, NaHCO₃ saturated solution, brine, dried over MgSO₄, filtered and the solvent was removed under reduced pressure. Product was purified by column chromatography (Pe:EtOAc 10:1) to give 330 mg (99%) of 2 as colourless oil. R_{f}=0.25 Pe:Et₂O 1:1. αD (CHCl₃, c=5.4)=−59.26°. IR (neat, cm⁻¹): 3513.81, 2955.02, 2927.36, 2856.57, 1783.55, 1697.94, 1455.31, 1386.51, 1351.64, 1210.50. ¹H NMR (400 MHz, CDCl₃): δ 7.36-7.17 (m, 5H), 4.75-4.65 (m, 1H), 4.26-4.16 (m, 2H), 3.98-3.89 (m, 1H), 3.76 (dq, 1H, J=7.0, 2.7 Hz), 3.25 (dd, 1H, J=13.4, 3.3 Hz), 2.84-2.74 (m, 2H), 1.35-1.22 (m, 12H), 1.25 (d, 3H, J=7.0 Hz), 0.87 (t, 3H, J=7.0 Hz); ¹³C NMR (100 MHz) δ 177.42, 152.98, 135.03, 129.36, 128.88, 127.33, 71.48, 67.85, 66.09, 55.04, 42.13, 37.72, 33.88, 31.74, 29.47, 25.95, 22.56, 14.00, 10.40. HRMS (El) ([M⁺]) Calcd. for C₂₁H₃₁NO₄Na: 384.2145. Found: 384.2198.

### (R)-4-Benzyl-3-((2S,3R)-3-fluoro-2-methyldecanoyl)oxazolidin-2-one (3)

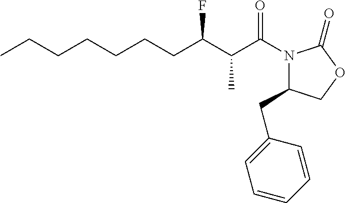 [SMILES: CCCCCCC[C@@H](F)[C@@H](C)C(=O)N1C(=O)OC[C@H]1Cc1ccccc1]

Aldol 2 (156 mg, 0.43 mmol) was diluted in anhydrous DCM (3 mL), cooled to −78° C., then a solution of DAST (57 μL, 0.43 mmol) in anhydrous DCM (2 mL) was added dropwise to the reaction mixture, stirred at −78° C. for 2 h, then allowed to reach ambient temperature. Reaction mixture was quenched by slow addition of water (5 mL). Organic layer was washed with NaHCO₃ saturated solution, brine, dried over MgSO₄, filtered and the solvent was removed under reduced pressure. Product was purified by column chromatography (Pe:EtOAc 10:1) to give 101 mg (64%) of 3 as a colourless oil. R_{f}=0.66 Pe:EtOAc 5:1. αD (CHCl₃, c=8.7)=−42.00°. IR (neat, cm⁻¹): 2926.84, 2857.15, 1781.04, 1699.78, 1497.88, 1455.61, 1391.80, 1350.87, 701.29. ¹H NMR (400 MHz, CDCl₃): δ 7.37-7.16 (m, 5H), 4.87-4.64 (m, 2H), 4.24-4.07 (m, 3H), 3.26 (dd, 1H, J=13.4, 3.3 Hz), 2.87-2.75 (m, 1H), 1.55-1.22 (m, 12H), 1.18 (d, 3H, J=7.0 Hz), 0.88 (t, 3H, J=6.8 Hz). ¹³C NMR (125 MHz) δ 174.32, 153.04, 135.15, 129.38, 128.86, 127.30, 94.81, 66.11, 55.32, 41.87, 37.79, 31.88, 31.70, 29.29, 29.07, 24.47, 22.55, 14.01, 13.58. ¹⁹F NMR (470 MHz) δ −179.69. HRMS (El) ([M⁺]) Calcd. for C₂₁H₃₀FNO₃Na: 386.2102. Found: 386.2096.

### (2S,3R)-3-Fluoro-2-methyldecanoic acid (4)

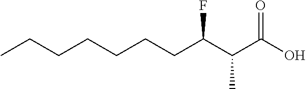

Oxazolidinone 3 (100 mg, 0.28 mmol), was diluted in THF (3 mL), cooled to 0° C., then 30% (v/v) H₂O_{2 aq} (0.14 mL, 1.65 mmol) and LiOH (13 mg, 0.55 mmol) were added and the reaction mixture was stirred at ambient temperature for 12 h. Reaction mixture was quenched by addition of Na₂SO_{3 aq} saturated solution (3 mL), extracted with DCM, organic layer was washed with water, brine, dried over MgSO₄, filtered and the solvent was removed under reduced pressure. Product was purified by column chromatography (Pe:EtOAc 5:1) to give 34 mg (61%) of 4 as a colourless oil. R_{f}=0.60 Pe:EtOAc 2:1. αD (CHCl₃, c=3.5)=+0.74°. ¹H NMR (400 MHz, CDCl₃): δ 10.56 (br s, 1H), 4.78-4.57 (m, 1H), 2.85-2.71 (m, 1H), 1.45-1.18 (m, 12H), 1.19 (d, 3H, J=7.2 Hz), 0.87 (t, 3H, J=7.0 Hz). ¹³C NMR (125 MHz) δ 179.91, 94.96, 93.59, 44.35, 31.74, 29.21, 24.80, 22.61, 14.06, 12.59, 12.54. ¹⁹F NMR (470 MHz) δ −181.96. HRMS (El) ([M⁺]) Calcd. for O₂₁H₃₀FNO₃Na: 386.2102. Found: 386.2096.

### (2S,3R)-3-Fluoro-2-methyldecanoic-CoA (5)

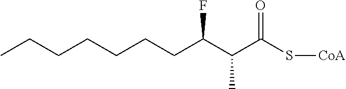

Acid 4 (10 mg, 0.05 mmol) was diluted in anhydrous DCM (1 mL), then CDI (14 mg, 0.09 mmol) was added in one portion and the reaction mixture was stirred at ambient temperature for 1 h. Reaction was washed with water (5×2 mL), brine, dried over MgSO₄, filtered and the solvent was removed under reduced pressure. Crude product was diluted in THF (1 mL) and then CoA-Li₃ (17 mg, 0.02 mmol) was added, followed by 0.1 M NaHCO₃ (1 mL) solution and the reaction mixture was stirred at ambient temperature for 18 h. Reaction was acidified to pH-3 with 1M HCl_{aq} and the solvents were partly removed under reduced pressure. Water (2 mL) was added and the mixture was extracted with EtOAc (5×3 mL). Aqueous layer was purified using solid phase extraction to give 6 mg of 5 as white powder. ¹H NMR (500 MHz, D₂O): δ 8.47 (s, 1H), 8.18 (s, 1H), 6.07 (d, 1H, J=7.1 Hz), 4.18-4.11 (m, 1H), 3.92 (s, 1H), 3.71-3.68 (m, 1H), 3.57 (d, 1H, J=4.6 Hz), 3.55 (d, 1H, J=4.6 Hz), 3.49-3.43 (m, 2H), 3.37-3.33 (m, 1H), 3.28-3.23 (m, 1H), 3.17 (t, 2H, J=6.5 Hz), 2.99-2.91 (m, 2H), 2.61 (t, 2H, J=6.5 Hz), 2.32 (t, 2H, J=6.5 Hz), 1.25-1.09 (m, 10H), 1.04 (d, 3H, J=7.1 Hz), 0.79 (s, 3H), 0.75 (t, 3H, J=6.9 Hz), 0.67 (s, 3H); ¹⁹F NMR (470 MHz) δ −181.08.

### Example 2: Synthesis of syn- and anti-3-fluoro-2-methyldecanoyl-CoA esters

An alternative reaction scheme for the preparation of compounds described herein was performed as follows:

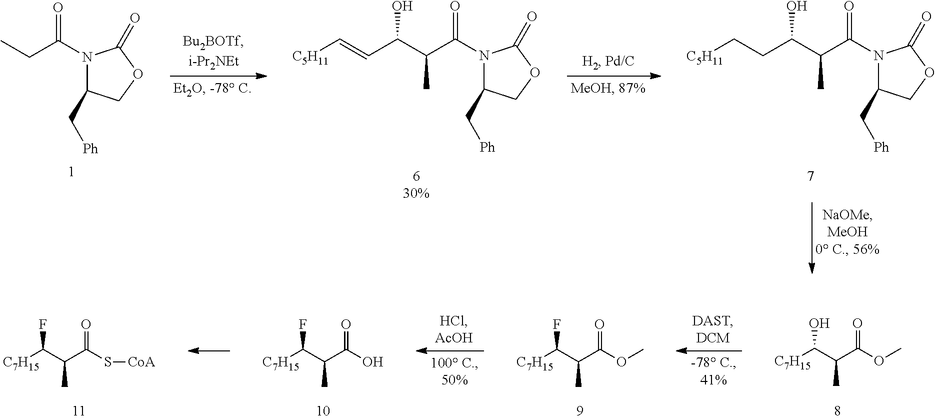

### (R)-4-Benzyl-3-[(2S,3S,E)-3-hydroxy-2-methyldec-4-enoyl]oxazolidin-2-one (6)

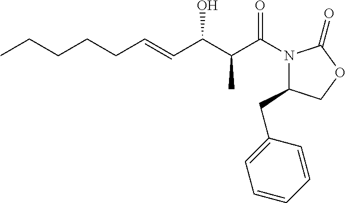 [SMILES: CCCCC/C=C/[C@H](O)[C@H](C)C(=O)N1C(=O)OC[C@H]1Cc1ccccc1]

A solution of dibutylboron triflate in DCM (1.0 M, 1.7 mL, 1.71 mmol) and diisopropylethylamine (0.17 mL, 0.99 mmol) were added to a stirred solution of oxazolidinone 1 (200 mg, 0.86 mmol) in 5 mL of Et₂O cooled to 0° C. and the resulting solution was stirred for 30 min. Reaction mixture was cooled to −78° C. and then 2-octenal (0.16 mL, 1.07 mmol) in 1 mL of Et₂O was added dropwise and the reaction mixture was stirred at −78° C. for 30 min and then allowed to reach ambient temperature. Reaction was quenched by slow addition of phosphate buffer (0.1 M, pH=7, 7 mL), organic layer was then washed with 1M HCl_{aq}, NaHCO₃ saturated solution, brine, dried over MgSO₄, filtered and the solvent was removed under reduced pressure. Product was purified by column chromatography (Pe:EtOAc 10:1) to give 92 mg (30%) of 6 as colourless oil. R_{f}=0.37 Pe:EtOAc 5:1. αD (CHCl₃, c=5.8)=+49.76°. IR (neat, cm⁻¹): 3497.60, 2957.31, 2927.41, 1780.02, 1698.19, 1454.87, 1387.30, 1351.35, 1211.34, 971.02, 706.32. ¹H NMR (400 MHz, CDCl₃): δ 7.37-7.20 (m, 5H), 5.83-5.69 (m, 1H), 5.56-5.46 (m, 1H), 4.74-4.64 (m, 1H), 4.27-4.12 (m, 3H), 3.98-3.89 (m, 1H), 3.30 (dd, 1H, J=13.5, 3.4 Hz), 2.83-2.72 (m, 1H), 2.51 (d, 1H, J=7.0 Hz), 2.10-2.01 (m, 2H), 1.44-1.23 (m, 6H), 1.16 (d, 3H, J=7.1 Hz), 0.87 (t, 3H, J=7.0 Hz). ¹³C NMR (125 MHz) δ 176.42, 153.49, 134.55, 129, 46, 128.92, 127.30, 75.88, 65.97, 55.47, 43.33, 37.80. 32.18, 31.35, 28.72, 22.45, 14.50, 13.98. HRMS (El) ([M⁺]) Calcd. for C₂₁H₃₁NO₄Na: 382.1994. Found: 382.2054.

### (R)-4-Benzyl-3-[(2S,3S)-3-hydroxy-2-methyldecanoyl]oxazolidin-2-one (7)

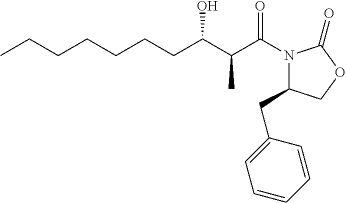 [SMILES: CCCCCCC[C@H](O)[C@H](C)C(=O)N1C(=O)OC[C@H]1Cc1ccccc1]

Aldol 6 (150 mg, 0.42 mmol) was diluted in EtOAc (10 mL), then Pd/C (15 mg) was added. Air was removed from the flask by syringe and replaced with hydrogen and the reaction mixture was stirred at ambient temperature for 24 h. Reaction was filtered through celite, washed with EtOAc and the solvent was removed under reduced pressure. Product was purified by column chromatography (Pe:EtOAc 5:1) to give 132 mg (87%) of 7 as a colourless oil. R₁=0.37 Pe:EtOAc 5:1. αD (CHCl₃, c=5.0)=−40.00°. IR (neat, cm⁻¹): 3524.14, 2927.23, 2856.31, 1779.94, 1698.07, 1455.08, 1386.53, 1351.39, 1210.51, 1107.51, 703.09. ¹H NMR (400 MHz, CDCl₃): δ 7.38-7.19 (m, 5H), 4.74-4.63 (m, 1H), 4.25-4.14 (m, 2H), 3.94-3.84 (m, 1H), 3.78-3.66 (m, 1H), 3.33 (dd, 1H, J=13.4, 3.3 Hz), 2.81-2.72 (m, 1H), 2.51 (d, 1H, J=8.6 Hz), 1.65-1.23 (m, 12H), 1.21 (d, 3H, J=6.8 Hz), 0.88 (t, 3H, J=7.0 Hz). ¹³C NMR (100 MHz) δ 176.84, 153.49, 135.21, 129.38, 128.89, 127.28, 74.61, 65.98, 55.48, 43.22, 37.82, 34.95, 31.75, 29.48, 29.18, 25.40, 22.57, 14.55, 14.01. HRMS (El) ([M⁺]) Calcd. for C₂₁H₃₁NO₄Na: 384.2145. Found: 384.2198.

### (2 S,3S)-Methyl 3-hydroxy-2-methyldecanoate (8)

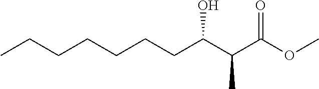

Sodium metal (48 mg, 2.08 mmol) was reacted with anhydrous MeOH (15 mL), cooled to 0° C., then a solution of aldol 7 (470 mg, 1.30 mmol) in anhydrous MeOH (5 mL) was added to the reaction mixture and stirred at 0° C. for 15 min. Reaction was quenched by slow addition of phosphate buffer (0.1 M, pH=7, 20 mL), extracted with DCM (4×20 mL), combined organic layer was washed with brine, dried over MgSO₄, filtered and the solvent was removed under reduced pressure. Product was purified by column chromatography (Pe:EtOAc 5:1) to give 158 mg (56%) of 8 as a colourless oil. R_{f}=0.67 Pe:EtOAc 5:1. αD (CHCl₃, c=10.2)=+5.88°. ¹H NMR (400 MHz, CDCl₃): δ 3.70 (s, 3H), 3.65 (br s, 1H), 2.58-2.44 (m, 2H), 1.60-1.22 (m, 12H), 1.20 (d, 3H, J=7.2 Hz), 0.87 (t, 3H, J=7.0 Hz). ¹³C NMR (100 MHz) δ 176.39, 73.32, 51.60, 45.11, 34.71, 31.73, 29.45, 29.16, 25.44, 22.56, 14.26, 13.99.

### (2R,3R)-Methyl 3-fluoro-2-methyldecanoate (9)

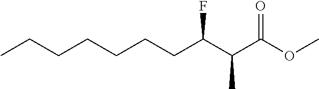

Compound 9 was prepared following the same procedure as for compound 3 from aldol 8 (54 mg, 0.25 mmol). Product was purified by column chromatography (Pe:EtOAc 20:1) to give 42 mg (78%) of 9 as a colourless oil. R_{f}=0.41 Pe:EtOAc 20:1. αD (CHCl₃, c=5.5)=+9.09°.¹H NMR (400 MHz, CDCl₃): δ 4.80-4.59 (m, 1H), 3.70 (s, 3H), 2.71-2.55 (m, 1H), 1.59-1.20 (m, 12H), 1.23 (d, 3H, J=7.1 Hz), 0.87 (t, 3H, J=7.0 Hz). ¹³C NMR (125 MHz) δ 174.05, 132.37, 94.75, 51.87, 44.28, 33.00, 29.11, 25.24, 22.61, 17.48, 14.06, 11.61. ¹⁹F NMR (470 MHz) δ −189.85.

### (2R,3R)-3-Fluoro-2-methyldecanoic acid (10)

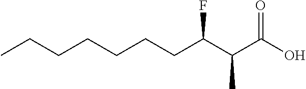

Methyl ester 9 (19 mg, 0.09 mmol) was diluted in glacial acetic acid (1 mL), then 12 M HCl_{aq} (1 mL) was added and the reaction mixture was heated at 100° C. for 4 h. Reaction mixture was cooled to ambient temperature and the solvents were removed under reduced pressure. Crude product was diluted in DCM, extracted with water twice and the organic layer was washed with brine, dried over MgSO₄, filtered and the solvent was removed under reduced pressure. Product was purified by column chromatography (Pe:EtOAc 2:1) to give 9 mg (50%) of 10 as a colourless oil. R_{f}=0.61 Pe:EtOAc 2:1. αD (CHCl₃, c=4.0)=+7.50°.¹H NMR (400 MHz, CDCl₃): δ 10.38 (br s, 1H), 4.86-4.64 (m, 1H), 2.73-2.59 (m, 1H), 1.53-1.19 (m, 15H), 0.88 (t, 3H, J=6.9 Hz). ¹⁹F NMR (470 MHz) δ −190.19.

### (2R,3R)-3-Fluoro-2-methyldecanoyl-CoA (11)

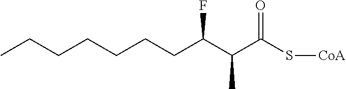

Compound 11 was prepared following the same procedure as for compound 5 from 4. ¹H NMR (500 MHz, D₂O): δ 8.71 (s, 1H), 8.48 (s, 1H), 6.30-6.20 (m, 1H), 4.67-4.58 (m, 1H), 4.36-4.24 (m, 2H), 4.11-4.02 (m, 1H), 3.96-3.85 (m, 1H), 3.70-3.57 (m, 1H), 3.55-3.34 (m, 2H), 3.15-2.99 (m, 2H), 2.59-2.40 (m, 2H), 2.31-2.17 (m, 1H), 1.84 (s, 2H), 1.50-1.16 (m, 10H), 0.97 (d, 3H, J=6.5 Hz), 0.86 (s, 3H), 0.84 (s, 3H); ¹⁹F NMR (470 MHz) δ −187.20.

### Example 3: AMACR Assays—Methods

Human AMACR was expressed in E. coli Rosetta2 (DE3) at 22° C. overnight shaking at 220 r.p.m.¹ Cells (˜2 g) were lysed using the ‘one shot’ in ˜30 mL buffer supplemented with 250u benzonase (Novagen) and stirred with 1.5% (w/v) N-lauroyl-sarcosine at 4° C. for 1 hour. Following centrifugation, enzyme was purified by metal-chelate chromatography, dialysed into 20 mM NaH₂PO₄—NaOH, pH 7.4 and stored at −80° C.^{1,2}

Assays were conducted in buffer containing ²H₂O and 100 μM acyl-CoA substrate as previously described, with negative controls contain heat-inactivated enzyme. ±-Fenoprofenoyl-CoA or 2-methyldecanoyl-CoA was used as known substrates.^{1,2} Assays were quenched by heating to 50° C. for 10 minutes before ¹H NMR analysis (500.13 MHz). Conversion of substrates was quantified by conversion of the 2-Me doublet at ca. 1.0 p.p.m. into a singlet.^{1,2} Kinetic data was analysed as previously described^{1,2} and parameters were obtained using SigmaPlot 12 with the Direct Linear Plot^{3,4} and non-linear fitting of Michaelis-Menten parameters.

The 3S and 3R stereoisomers of the 3-fluoro-2-methylacyl-CoA esters were then incubated with AMACR and the reaction was monitored by ¹H and ¹⁹F NMR. Interconversion of 3S and 3R by chiral inversion was expected to be observed. Instead an elimination reaction occurred, in which both 3S and 3R were converted into the 2-unsaturated 2-methylacyl-CoA ester and inorganic fluoride; the ¹H NMR spectrum of the acyl-CoA product was identical to that of authentic synthetic 2-unsaturated 2-methylacyl-CoA ester. The synthetic product compound was produced from the E-acid, which had the alkene proton at 6.92 p.p.m., whilst the Z-isomer has an alkene proton at 6.09 p.p.m. in the ¹H NMR spectrum. The presence of fluoride was observed as a characteristic signal at −120 p.p.m. in the ¹⁹F spectrum. Increasing amounts of the unsaturated product compound and inorganic fluoride were observed upon incubation of either 3S or 3R with AMACR for longer times. This reaction was not observed in negative controls containing heat-inactivated enzyme. Incubation of the synthetic unsaturated product compound in the presence of fluoride did not show any conversion to the 3-fluoro-2-methylacyl-CoA esters. This observed irreversibility of the elimination reaction probably results from the high levels of hydration of the fluoride anion.

Both 3S and 3R give the same unsaturated product, consistent with an E1cb mechanism in which the enolate intermediate is used to expel the fluoride. The resulting E-double bond suggests the reaction occurs with the substrate in an anti-conformation with respect to the α-proton and fluoride. This contrasts with the syn-reaction of enoyl-CoA hydratase and crotonase enzymes, which results from the two catalytic glutamate residues been on the same face of the substrate. The AMACR-catalysed elimination differs in several crucial respects: Firstly, the substrate side-chain is bound by a hydrophobic surface rather than a defined binding pocket; secondly, fluorine is a small moiety comparable in size to hydrogen; and third, fluoride is a much better leaving group than the hydroxide anion, and therefore does not require protonating for the elimination reaction to occur. Moreover, the fluoride anion will be highly solvated in water. It is likely these factors allow the elimination reaction to proceed with anti-stereochemistry.

The following kinetic parameters were determined for AMACR by the Direct Linear Plot with R isomer starting material: Kₘ=21 μM; Vₘₐₓ=96.5 nmol·min⁻¹·mg polypeptide⁻¹; k_{cat}=0.0758 s⁻¹; k_{cat}/Kₘ=3612 M⁻¹ s⁻¹. This compares to Kₘ=277 μM; Vₘₐₓ=39.3 nmol·min⁻¹·mg⁻¹ polypeptide; k_{cat}=0.0310 s⁻¹; k_{cat}/Kₘ=112 M⁻¹ s⁻¹ for S-2-methyldecanoyl-CoA, implying that the elimination reaction is −32× more efficient than the chiral inversion reaction (as judged by k_{cat}/Kₘ). This comparatively high catalytic efficiency is likely to be related to the increased acidity of the α-proton in 5 due to the electron-withdrawing effect of the fluorine atom.⁹ For 11, significant background conversion was observed in negative controls, making it difficult to measure kinetic parameters, probably due to the anti-arrangement of the α-proton and fluorine atom in the molecule facilitating non-enzymatic elimination. However, the following approximate values were obtained: Kₘ=40 μM; Vₘₐₓ=50.6 nmol·min⁻¹·mg polypeptide⁻¹; k_{cat}=0.0397 s⁻¹; K_{cat}/kₘ=993 M⁻¹ s⁻¹.

The results demonstrate that human AMACR is able to catalyse the elimination of substrates, probably by an E1cb or E1cb-like E2 mechanism.

### Example 4: Measurement of Inhibitor Potency Using the Elimination Assay

Assays were performed as previously described in a total volume of 0.55 mM in the presence of ²H₂O. using human recombinant AMACR 1A². In the following table inhibitor at 100 μM was incubated with enzyme for 10 minutes at room temperature. The reaction was initiated by addition of a stock solution of substrate (2S,3R-3-fluoro-2-methyldecanoyl-CoA) to a final concentration of 100 μM, followed by incubation at 30° C. for 1 hour. The reaction was terminated by heating at ca. 55° C. for 10 minutes. The cooled reaction mixture analysed by ¹H NMR by the appearance of the unsaturated product peak at ca. 1.75 ppm and comparison to the residual substrate peak at 1.1 ppm.⁵ Assays were compared to a positive control (no inhibitor) and conversion levels are corrected for levels in negative controls (boiled enzyme). The data in the table is the average of two dependent repeats.

| | | | Average |
|---|---|---|---|
| Potential | | Enzyme | reduction in |
| inhibitor | Structure | (mg/mL) | conversion |
| Fenoprofenoyl- CoA | 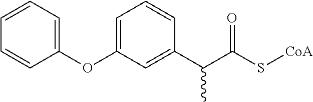 | 0.06 | 14.1% |
| Ibuprofenoyl- CoA | 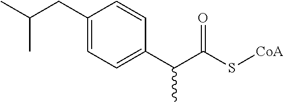 | 0.06 | 14.5% |
| Ketoprofenoyl- CoA | 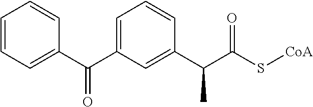 | 0.06 | 4.5% |
| Carnell's most potent compound⁶ | 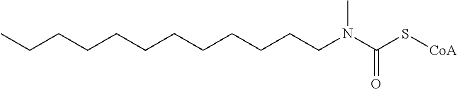 | 0.06 | 46.8% |
| Naproxenoyl-CoA | 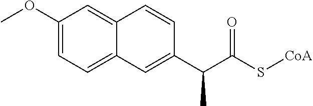 | 0.06 | 4.7% |
| Flurbiprofenoyl- CoA | 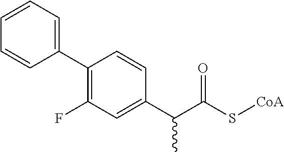 | 0.06 | 3.5% |

Similar assays were also conducted where inhibitor and substrate were added to the enzyme simultaneously.

| | | | Average |
|---|---|---|---|
| Potential | | Enzyme | reduction in |
| inhibitor | Structure | (mg/mL) | conversion |
| Ibuprofenoyl- CoA | 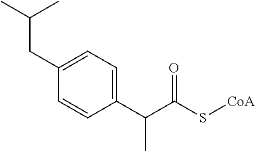 | 0.12 | 11% |
| R-2- methyldecanoyl- CoA | 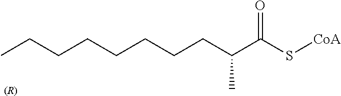 | 0.12 | 13.6% |
| S-2- methyldecanoyl- CoA | 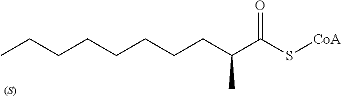 | 0.12 | 7.2% |

All of the above listed potential inhibitors (in both tables) were prepared using established methods.^{1,6,9}

### Example 5: A Colorimetric Assay for AMACR

Based on this elimination reaction, a colorimetric assay was developed. A colorimetric substrate was proposed using nitrophenol derivatives, which upon elimination produce an intense yellow colour. 2,4-dintrophenol was chosen as the leaving group as the pKa of the acid is similar to that of HF.

The required substrate was synthesised by aromatic substitution of 2,4-dinitro-1-fluorobenzene by diol. The resulting product was oxidised to the acid, which was converted to the required acyl-CoA using established methods.^{5,7}

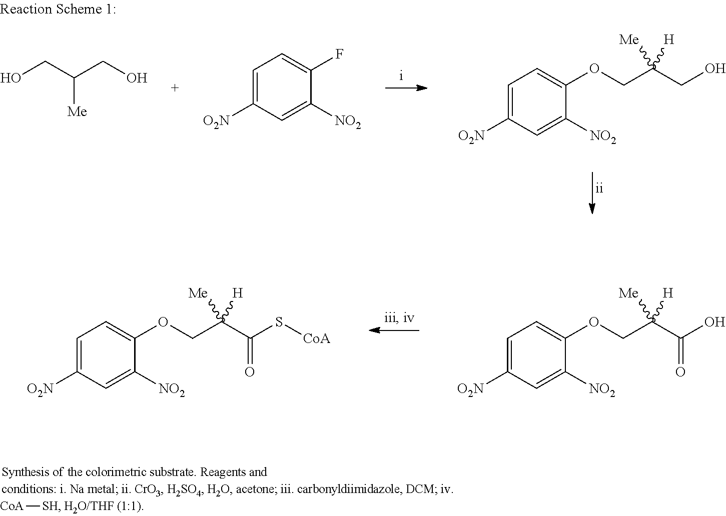

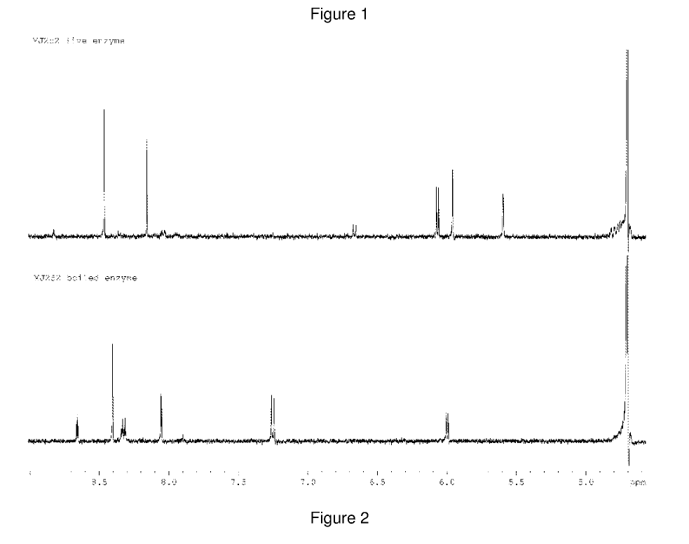

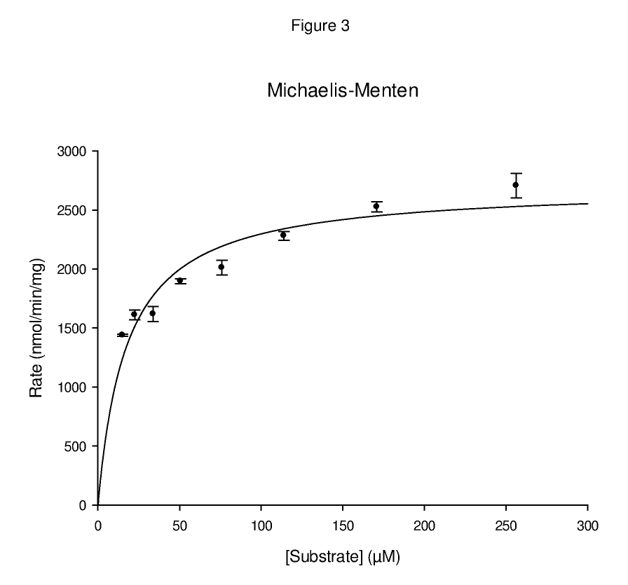

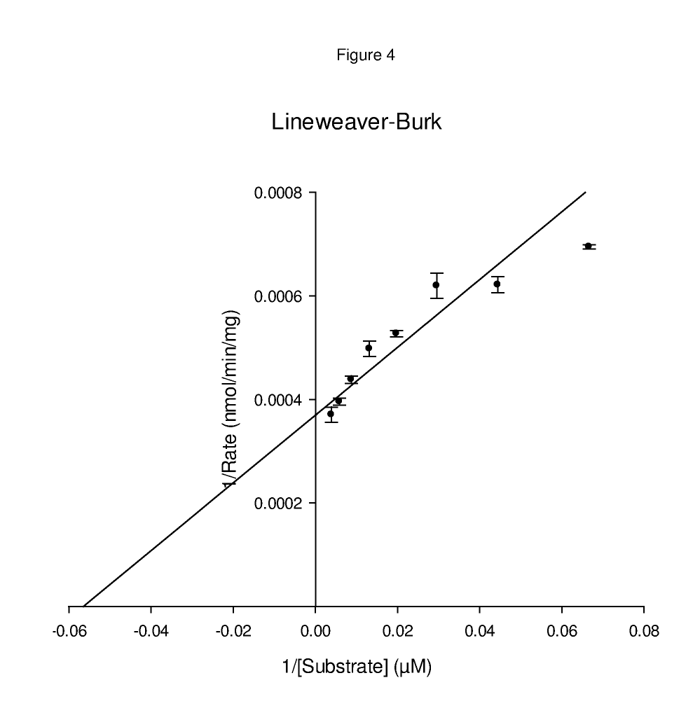

Incubation of the substrate with active AMACR resulted in an intense yellow colour been produced, which was absent in negative controls using heat-inactivated enzyme. Analysis of the reaction mixture by ¹H NMR showed that an elimination reaction had occurred (see FIG. 1 as compared to the negative control FIG. 2). The elimination reaction is shown in Reaction Scheme 2 below:

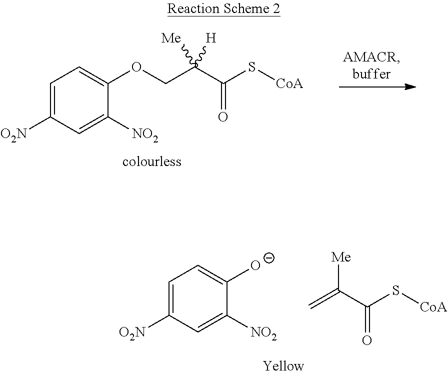

The above assay was transferred into a 96-well plate format with the extent of reaction monitored by absorbance at 354 nm. The reaction was carried out in Na₂HPO₄—NaOH, pH 7.4 in a total volume of 100 μL containing 8.05 μg (0.17 nmoles) of human recombinant AMACR 1A. Kinetic parameters were calculated using an extinction coefficient of 15, 300 M⁻¹ cm⁻¹ as set out Chafaa et al.⁸ The following kinetic parameters were determined for the enzyme: Kₘ=18 μM; Vₘₐₓ=27.61 nmol·min⁻¹ mg⁻¹; k_{cat}=0.023 s⁻¹; k_{cat}/Kₘ=1278 M⁻¹ s⁻¹. The substrate is converted ˜35% as efficiently as anti-3-fluoro-2-methyldecanoyl-CoA, as judged by k_{cat}/Kₘ.⁵

The kinetics assay was carried out in a 96 well plate. Substrate stock solutions (at 2× the final concentration) were prepared by serial dilution in a total volume of 150 μL in phosphate buffer (as already described). This solution (50 μL) was transferred into two adjacent wells, such that three identical wells were prepared. The reaction was initiated by adding a 2× stock solution of enzyme (50 μL per well) to the substrate, mixing and measuring the change in absorbance at 354 nm over a 15 minute time course, with data collected every 60 seconds. The reaction was conducted at ambient room temperature, ca. 25° C. The final concentrations of substrate (in the assay, after adding the enzyme) were: 256.3, 180.9, 113.9, 75.9, 50.6, 33.8, 22.5, 15.0 μM.

Rates were determined by plotting the absorbance vs. time in Microsoft Excel. The initial rate was used before the rate starts to reduce due to substrate depletion and other reasons. The rate in absorbance units/min was derived by fitting a straight line to the points and determining the slope from the y=mx+c equation.

The data was converted into nmol/min/mg using the extinction coefficient,⁸ the pathlength (calculated by the plate-reader based on the volume of the reaction; 0.33 cm), and the amount of enzyme determined by UV-visible spectroscopy.¹ Kinetic constants were derived using SigmaPlot 12 with the enzyme kinetics macro function. Fitting assumed a single substrate per active site, two active sites per dimer (one active sites per protein molecule), with three repeats of each rate. Data was fitted to the standard Michaelis-Menten and substrate inhibition models and graphs showed that the Michaelis-Menten model was correct. Kinetic data was also calculated using the Direct Linear Plot^{3,4} A similar method (with a different enzyme) has also been described.¹⁰

### REFERENCES

1 T. J. Woodman, P. J. Wood, A. S. Thompson, T. J. Hutchings, G. R. Steel, P. Jiao, M. D. Threadgill, and M. D. Lloyd, Chem. Commun., 2011, 47, 7332-7334.
2 D. J. Darley, D. S. Butler, S. J. Prideaux, T. W. Thornton, A. D. Wilson, T. J. Woodman, M. D. Threadgill, and M. D. Lloyd, Org. Biomol. Chem., 2009, 7, 543-552.
3 A. Cornish-Bowden, and R. Eisenthal, Biochem. J., 1974, 139, 721-730.
4 R. Eisenthal, and A. Cornish-Bowden, Biochem. J., 1974, 139, 715-720.
5 Yevglevskis, M., Threadgill, M. D., Woodman, T. J. and Lloyd, M. D., Chem. Commun., 2014, 50, 14164-14166.
6 Carnell, A. J., Kirk, R., Smith, M., McKenna, S., Lian, L.-Y. and Gibson, R., ChemMedChem., 2013, 8, 1643-1647.
7 Yevglevskis, M., Bowskill, C. R., Chan, C. C. Y., Heng, J. H.-J., Threadgill, M. D., Woodman, T. J. and Lloyd, M. D., Org. Biomol. Chem., 2014, 12, 6737-6744.
8 Chafaa, S., Meullemeestre, J., Schwing, M. J., Vierling, F., Bohmer, V. and Vogt, W., Helv. Chim. Acta., 1993, 76, 1425-1434.
9 Carnell, A. J., Hale, I., Denis, S., Wanders, R. J. A., Isaacs, W. B., Wilson, B. A. and Ferdinandusse, S. (2007) Journal of Medicinal Chemistry, 2007, 50, 2700-2707.
10 Qu, X., Allan, A., Chui, G., Hutchings, T. J., Jiao, P., Johnson, L., Leung, W. Y., Li, P. K., Steel, G. R., Thompson, A. S., Threadgill, M. D., Woodman, T. J., and Lloyd, M. D., Biochem. Pharmacol. 2013, 86, 1621-1625.

Any publication cited or described herein provides relevant information disclosed prior to the filing date of the present application. Statements herein are not to be construed as an admission that the inventors are not entitled to antedate such disclosures. All publications mentioned in the above specification are herein incorporated by reference. Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology and molecular biology or related fields are intended to be within the scope of the following claims.

1. A method of measuring α-methylacyl-CoA racemase (AMACR) activity in a sample comprising the steps of:
providing a sample;
(ii) contacting said sample with a substrate compound that undergoes elimination of hydrogen and a leaving group catalysed by said AMACR to produce a product compound having an additional carbon-carbon double bond; and
(iii) measuring the amount of said leaving group and/or of said product compound and/or of unreacted substrate compound.

2. The method according to claim 1, wherein said hydrogen and said leaving group in said substrate compound are bonded to adjacent carbon atoms, whereby said elimination results in formation of a carbon-carbon double bond between said adjacent carbon atoms.

3. The method according to claim 1, wherein said hydrogen in said substrate is bonded to a carbon atom further having methyl and carbonyl substituents, preferably wherein said carbonyl substituent is selected from —COOCoA, —COSCoA, —CONHCoA, —COCH₂SCoA, or —COCH₂OCoA.

4. The method according to claim 1, wherein said leaving group is fluorine.

5. The method according to claim 1, wherein said carbon-carbon double bond is conjugated to one or more unsaturated moieties in said product compound.

6. The method according to claim 1, wherein said step of measuring comprises NMR spectroscopy, or measuring UV/visible absorption or fluorescence of said leaving group or a derivative thereof, and/or of said product compound and/or of said unreacted substrate compound, or measuring circular dichroism or optical rotation.

7. The method according to claim 1, wherein said substrate compound has formula I: 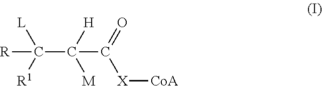
wherein:
L is the leaving group;
M is hydrogen, a methyl group or a methyl-mimetic group;
R¹ is H or an R group as defined below;
X is O, S, CH₂, C1-C7 alkyleneoxy, C1-C7 alkylenethio or NH; and
R is a group selected from substituted or unsubstituted alkyl, aryl, (hetero)alkyl, (hetero)alkenyl, (hetero)alkynyl, (hetero)aryl, arylalkyl, (hetero)arylalkyl, cycloalkyl, (hetero)cyclyl, cycloalkylaryl, (hetero)cycloalkyl, (hetero)cycloalkylaryl, heterocyclylalkyl, a peptide, an oligosaccharide, a steroid group or H.

8. The method according to claim 7, wherein M is H or methyl.

9. The method according to claim 7, wherein the leaving group is fluorine or a substituted or unsubstituted mono-nitrophenoxide, di-nitrophenoxide or tri-nitrophenoxide group.

10. The method according to claim 7, wherein L is F, M is methyl, and R¹ is H or C1-C7 alkyl.

11. The method according to claim 1, wherein said sample is a biological sample removed from a subject.

12. A method for diagnosing and/or detecting a disease in a subject, comprising the steps of:
(i) providing a sample from a subject;
(ii) measuring the AMACR activity in the sample according to the method of claim 1;
(iii) comparing the measurement from step (ii) with a reference standard; and
(iv) using the comparative measurement from step (iii) to determine whether the subject has a disease;
(v) optionally, treating said subject if said subject has the disease.

13. The method according to claim 12, wherein the disease is cancer or Anisakisis.

14. A method of monitoring the metastasis of a cancer in a subject, the method comprising the steps:
(i) providing samples from a subject obtained at first and second time points;
(ii) measuring the AMACR activity in the samples according to the method of claim 1; and
(iii) comparing the at least two measurements from step (ii) with each other;
wherein an increase in AMACR activity in the sample taken at the later time point compared to the corresponding level of AMACR activity in the sample taken at the earlier time point is indicative of an increase in the number of circulating cancer cells in the patient, and wherein a decrease in the level of AMACR activity in the sample taken at the later time point compared the corresponding level of AMACR activity in the sample taken at the earlier time point is indicative of a decrease in the number of circulating cancer cells in the patient;
(iv) optionally, treating said subject if said subject has metastatic cancer.

15. A method of monitoring the effectiveness of an anti-cancer therapy in a subject, the method comprising the steps:
providing samples from a subject obtained at first and second time points, wherein the subject has been treated with an anti-cancer therapy before the two time points or in the interval between the first and second time points, and
(ii) measuring the AMACR activity in the samples according to the method of claim 1; and
(iii) comparing the at least two measurements from step (ii);
wherein a decrease in the level of AMACR activity in the sample taken at the later time point compared the corresponding level of AMACR activity in the sample taken at the earlier time point is indicative of the anti-cancer therapy being efficacious, and wherein an increase in the level of AMACR activity in the sample taken at the later time point compared the corresponding level of AMACR activity in the sample taken at the earlier time point is indicative of the anti-cancer therapy being non-efficacious.

16. A method of assaying for the presence of a biomarker which is indicative of cancer cells in a sample, the method comprising the steps:
(i) providing a sample from a subject;
(ii) measuring the AMACR activity in the sample according to the method of claim 1; and
(iii) comparing the measurement from step (ii) with a reference standard;
wherein an increase in AMACR activity in the biological sample as compared to the reference standard is indicative of cancer cells being present in the biological sample.

17. A compound of Formula I, wherein said compound undergoes elimination of H and the leaving group L catalysed by AMACR to produce a product compound of formula II: 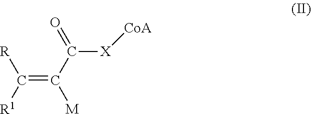
or an E/Z isomer thereof, wherein R, M and X are as defined in relation to Formula (I).

18. The compound according to claim 17 for use in a method of diagnosis according to claim 13.

19. The compound according to claim 17 for use in a method of diagnostic imaging of AMACR activity in a subject, comprising: administering said compound to a subject, followed by imaging the distribution of said leaving group and/or of said product compound and/or of unreacted substrate compound in the subject.

20. A method for identifying a compound that modulates the activity of AMACR comprising the steps of:
(i) providing a sample comprising AMACR;
(ii) providing at least one test compound;
(iii) contacting said sample in the presence and absence of said test compound with a substrate compound that undergoes elimination of hydrogen and a leaving group catalysed by said AMACR to produce a product compound having an additional carbon-carbon double bond; and
(iv) measuring the amount of said leaving group and/or of said product compound and/or of unreacted substrate compound in the presence and absence of said test compound,
wherein a change in the amount of said leaving group and/or of said product compound and/or of unreacted substrate compound in the presence and absence of said test compound is indicative that said test compound modulates the activity of AMACR.

21. A kit for measuring AMACR activity in a sample comprising:
(i) a substrate compound that undergoes elimination of hydrogen and a leaving group catalysed by AMACR to produce a product compound having an additional carbon-carbon double bond or the substrate compound according to claim 17;
(ii) a positive control that reacts with the substrate compound that is indicative of AMACR activity; and/or
(iii) a negative control that does not react with the substrate compound.

22. Use of a substrate compound that undergoes elimination of hydrogen and a leaving group catalysed by AMACR to produce a product compound having an additional carbon-carbon double bond or the use of the substrate compound according to claim 17 for measuring α-AMACR activity in a sample.

23. Use of a substrate compound that undergoes elimination of hydrogen and a leaving group catalysed by AMACR to produce a product compound having an additional carbon-carbon double bond or the use of the substrate compound according to claim 17 for diagnosing a disease.

24. Use of a substrate compound that undergoes elimination of hydrogen and a leaving group catalysed by AMACR to produce a product compound having an additional carbon-carbon double bond or the use of the substrate compound according to claim 17 for the diagnostic imaging of AMACR activity.


SEQUENCE LISTING (17 sequences)

SEQ ID NO: 1 - DNA, 3352 bp, Homo sapiens
ggggcgtggcgccggggattgggagggcttcttgcaggctgctgggctggggctaagggctgctcagtttccttcagcggggcactgggaagcgccatggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtgttctctcaaaaattggcagaagtggtgagaatccgtatgccccgctgaatctcctggctgactttgctggtggtggccttatgtgtgcactgggcattataatggctctttttgaccgcacacgcactggcaagggtcaggtcattgatgcaaatatggtggaaggaacagcatatttaagttcttttctgtggaaaactcagaaattgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctgatgaacttcccaatcagatgagcatggatgattggccagaaatgaagaagaagtttgcagatgtatttgcagagaagacgaaggcagagtggtgtcaaatctttgacggcacagatgcctgtgtgactccggttctgacttttgaggaggttgttcatcatgatcacaacaaggaacggggctcgtttatcaccagtgaggagcaggacgtgagcccccgccctgcacctctgctgttaaacaccccagccatcccttctttcaaaagggatcctttcataggagaacacactgaggagatacttgaagaatttggattcagccgcgaagagatttatcagcttaactcagataaaatcattgaaagtaataaggtaaaagctagtctctaacttccaggcccacggctcaagtgaatttgaatactgcatttacagtgtagagtaacacataacattgtatgcatggaaacatggaggaacagtattacagtgtcctaccactctaatcaagaaaagaattacagactctgattctacagtgatgattgaattctaaaaatggttatcattagggcttttgatttataaaactttgggtacttatactaaattatggtagttattctgccttccagtttgcttgatatatttgttgatattaagattcttgacttatattttgaatgggttctagtgaaaaaggaatgatatattcttgaagacatcgatatacatttatttacactcttgattctacaatgtagaaaatgaggaaatgccacaaattgtatggtgataaaagtcacgtgaaacagagtgattggttgcatccaggccttttgtcttggtgttcatgatctccctctaagcacattccaaactttagcaacagttatcacactttgtaatttgcaaagaaaagtttcacctgtattgaatcagaatgccttcaactgaaaaaaacatatccaaaataatgaggaaatgtgttggctcactacgtagagtccagagggacagtcagttttagggttgcctgtatccagtaactcggggcctgtttccccgtgggtctctgggctgtcagctttcctttctccatgtgtttgatttctcctcaggctggtagcaagttctggatcttatacccaacacacagcaacatccagaaataaagatctcaggaccccccagcaagtcgttttgtgtctccttggactgagttaagttacaagcctttcttatacctgtctttgacaaagaagacgggattgtctttacataaaaccagcctgctcctggagcttccctggactcaacttcctaaaggcatgtgaggaaggggtagattccacaatctaatccgggtgccatcagagtagagggagtagagaatggatgttgggtaggccatcaataaggtccattctgcgcagtatctcaactgccgttcaacaatcgcaagaggaaggtggagcaggtttcttcatcttacagttgagaaaacagagactcagaagggcttcttagttcatgtttcccttagcgcctcagtgattttttcatggtggcttaggccaaaagaaatatctaaccattcaatttataaataattaggtccccaacgaattaaatattatgtcctaccaacttattagctgcttgaaaaatataatacacataaataaaaaaatatatttttcatttctatttcattgttaatcacaactacttactaaggagatgtatgcacctattggacactgtgcaacttctcacctggaatgagattggacactgctgccctcattttctgctccatgttggtgtccatatagtacttgattttttatcagatggcctggaaaacccagtctcacaaaaatatgaaattatcagaaggattatagtgcaatcttatgttgaaagaatgaactacctcactagtagttcacgtgatgtctgacagatgttgagtttcattgtgtttgtgtgttcaaatttttaaatattctgagatactcttgtgaggtcactctaatgccctgggtgccttggcacagttttagaaataccagttgaaaatatttgctcaggaatatgcaactaggaaggggcagaatcagaatttaagctttcatattctagccttcagtcttgttcttcaaccatttttaggaactttcccataaggttatgttttccagcccaggcatggaggatcacttgaggccaagagttcgagaccagcctggggaacttggctggacctccgtttctacgaaataaaaataaaaaaattatccaggtatggtggtgtgtgcctgtagtcctatctactcaagggtggggcaggaggatcacttgagcccaggaatttgaggccacagtgaattaggattgcaccactgcactctagcccaggcaacagaacaagaacctgtctctaaataaataaataaaaataataataataaaaaagatgttttccctacaa

SEQ ID NO: 2 - protein, 382 aa, Homo sapiens
MALQGISVVELSGLAPGPFCAMVLADFGARVVRVDRPGSRYDVSRLGRGKRSLVLDLKQPRGAAVLRRLCKRSDVLLEPFRRGVMEKLQLGPEILQRENPRLIYARLSGFGQSGSFCRLAGHDINYLALSGVLSKIGRSGENPYAPLNLLADFAGGGLMCALGIIMALFDRTRTGKGQVIDANMVEGTAYLSSFLWKTQKLSLWEAPRGQNMLDGGAPFYTTYRTADGEFMAVGAIEPQFYELLIKGLGLKSDELPNQMSMDDWPEMKKKFADVFAEKTKAEWCQIFDGTDACVTPVLTFEEVVHHDHNKERGSFITSEEQDVSPRPAPLLLNTPAIPSFKRDPFIGEHTEEILEEFGFSREEIYQLNSDKIIESNKVKASL

SEQ ID NO: 3 - protein, 382 aa, Homo sapiens; identical to SEQ ID NO: 2

SEQ ID NO: 4 - protein, 281 aa, Homo sapiens
MALQGISVVELSGLAPGPFCAMVLADFGARVVRVDRPGSRYDVSRLGRGKRSLVLDLKQPRGAAVLRRLCKRSDVLLEPFRRGVMEKLQLGPEILQRENPRLIYARLSGFGQSGSFCRLAGHDINYLALSGGRNSIFKFFSVENSEIESVGSTSRTEHVGWWSTFLYDLQDSRWGIHGCWSNRTPVLRAADQRSGRTDLAENTTGSSIVPGKANPRQCEAMAIVAAQVMGFCVAVTVGGGNGHFELDVFKPMMIKNLCYTQAAGGCFSFLHRKLNSGNPDQ

SEQ ID NO: 5 - protein, 260 aa, Homo sapiens
MALQGISVVELSGLAPGPFCAMVLADFGARVVRVDRPGSRYDVSRLGRGKRSLVLDLKQPRGAAVLRRLCKRSDVLLEPFRRGVMEKLQLGPEILQRENPRLIYARLSGFGQSGSFCRLAGHDINYLALSGVLSKIGRSGENPYAPLNLLADFAGGGLMCALGIIMALFDRTRTGKGQVIDANMVEGTAYLSSFLWKTQKLSLWEAPRGQNMLDGGAPFYTTYRTADGEFMAVGAIEPQFYELLIKGCWGMLQFPSQKTK

SEQ ID NO: 6 - protein, 288 aa, Homo sapiens
MALQGISVVELSGLAPGPFCAMVLADFGARVVRVDRPGSRYDVSRLGRGKRSLVLDLKQPRGAAVLRRLCKRSDVLLEPFRRGVMEKLQLGPEILQRENPRLIYARLSGFGQSGSFCRLAGHDINYLALSGVLSKIGRSGENPYAPLNLLADFAGGGLMCALGIIMALFDRTRTGKGQVIDANMVEGTAYLSSFLWKTQKLSLWEAPRGQNMLDGGAPFYTTYRTADGEFMAVGAIEPQFYELLIKGLGELILLKIQQEAVSCQARQTLVSVKQWPSLQPKSWGFVWQ

SEQ ID NO: 7 - protein, 284 aa, Homo sapiens
MTKSSLRPLQKQSPQTGGLPPDCSKCCHGDYSFRGYQGPPGPPGPPGIPGNHGNNGNNGATGHEGAKGEKGDKGDLGPRGERGQHGPKGEKGYPGIPPELQIAFMASLATHFSNQNSGIIFSSVETNIGNFFDVMTGRFGAPVSGVYFFTFSMMKHEDVEEVYVYLMHNGNTVFSMYSYEMKGKSDTSSNHAVLKLAKGDEVWLRMGNGALHGDHQRFSTFAGFLLFETKCHGETPAGPRDSAAGKSKAYLCQAEWIWPVRKLLPVSWPRYQLFGFVRWKEQHI

SEQ ID NO: 8 - protein, 198 aa, Homo sapiens [sequence omitted]

SEQ ID NO: 9 - protein, 198 aa, Homo sapiens [sequence omitted]

SEQ ID NO: 10 - protein, 360 aa, Mycobacterium tuberculosis [sequence omitted]

SEQ ID NO: 11 - protein, 418 aa, Anisakis simplex
MAATTTVCSVCSRRFLSQILLISRRFSSSGGSSEAFEGSSSSSESATSQSQDSKLSDGVPIPKKPPLHGLKVIELSGLPAIPTGVAVVPYCGLILADFGAEVTVVKKAEHELLPYRLARGKTIKVVNLERAKDNKHVRNMCLESDVLLDPYRPGVIEKVGLNPLELLKENEKLIVARITGFGQTGELSQRFGRELNYVALSGLMPTFAGHSVKRFPYWTPANMLAGFAGGSLMAAFGILAAIHQRIINGGKGSIIDVSMVDGLAYLGSFQTIYKDIDNVWNKPFSWFSGDCPVYRCYETSDGKFMAVAALERRFHTKMLQVLEVNIPVSDIWTKPAEVTEKLEAVFKTKTRREWMEIFRGVDACVTPVVGLNEVGRIRHHQQRKSFKLEGHKFIPQPTPRIYSCEEYEAMLDSTKLKI

SEQ ID NO: 12 - DNA, 1149 bp, Homo sapiens
atggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtgttctctcaaaaattggcagaagtggtgagaatccgtatgccccgctgaatctcctggctgactttgctggtggtggccttatgtgtgcactgggcattataatggctctttttgaccgcacacgcactggcaagggtcaggtcattgatgcaaatatggtggaaggaacagcatatttaagttcttttctgtggaaaactcagaaatcgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctgatgaacttcccaatcagatgagcatggatgattggccagaaatgaagaagaagtttgcagatgtatttgcaaagaagacgaaggcagagtggtgtcaaatctttgacggcacagatgcctgtgtgactccggttctgacttttgaggaggttgttcatcatgatcacaacaaggaacggggctcgtttatcaccagtgaggagcaggacgtgagcccccgccctgcacctctgctgttaaacaccccagccatcccttctttcaaaagggatcctttcataggagaacacactgaggagatacttgaagaatttggattcagccgcgaagagatttatcagcttaactcagataaaatcattgaaagtaataaggtaaaagctagtctctaa

SEQ ID NO: 13 - DNA, 597 bp, Homo sapiens
atggcactgcagggcatctcggtcatggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtggaaggaacagcatatttaagttcttttctgtggaaaactcagaaatcgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctag

SEQ ID NO: 14 - DNA, 864 bp, Homo sapiens
atggccctgcaagggacatccgtggttgagctctctgggttggcccccggacctttctgtgccatggtactggctgacttcggcgctcgggttgtacgcgttgaccggccaggctcccggtatgacgtgagccgattggggcgaggtaaacgttcgcttgtactggatctcaagcaacctcggggggccgcggttctacgaaggctatgcaaaaggagcgatgtgctattggagccctttcgtaggggagtgatggaaaagttacagctaggaccggagatcttgcagcgagaaaacccaagacttatctatgcaagactaagtggatttggtcaaagcggaagtttttgtcgtttagccggccatgatattaattatctggcgcttagcggtgtcctttcaaagataggtagaagtggagaaaatccatacgcacctctcaacttattggctgatttcgcggggggagggctcatgtgcgctctggggattataatggcactcttcgatcgcactcgtacaggcaaagggcaagtcattgacgccaacatggtcgagggcaccgcttacctttcgagttttctgtggaaaacgcagaagtcttcattatgggaggctccgagaggtcaaaatatgctagacggtggagcgccgttttatacaacttacaggaccgcagatggtgaattcatggcagtgggtgcaatcgaaccccagttttacgaattactaataaagggcctcggcgagcttattttattgaagatacaacaggaagcggtttcgtgtcaggcgcgccagacgttggtatccgtaaaacagtggccatctttacaacctaaatcatggggcttcgtctggcaa

SEQ ID NO: 15 - DNA, 846 bp, Homo sapiens
atggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtggaaggaacagcatatttaagttcttttctgtggaaaactcagaaattgagtctgtgggaagcacctcgaggacagaacatgttggatggtggagcacctttctatacgacttacaggacagcagatggggaattcatggctgttggagcaatagaaccccagttctacgagctgctgatcaaaggtctgggagaactgatcttgctgaagatacaacaggaagcagtatcgtgccaggcaaggcaaaccctcgtcagtgtgaagcaatggccatcgttgcagcccaagtcatggggttttgtgtggcagtaaccgttggaggcggcaatggccattttgagttggatgttttcaagccaatgatgattaaaaatttatgttacactcaggctgctgggggatgcttcagtttccttcacagaaaactaaatagtgggaatccggaccaataa

SEQ ID NO: 16 - DNA, 773 bp, Homo sapiens
atggcactgcagggcacctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtgttctctcaaaaattggcagaatccgtatgccccgctgaatctcctggctgactttgctggtggtggccttatgtgtgcactgggcattataatggctctttttgaccgcacacgcactggcaagggtcaggtcattgatgcaaatatggtggaaggaacagcatatttaagttcttttctgtggaaaactcagaaattgagtctgtgggaagcacctcgaggacagaacatgttggatggtggagcacctttctatacgacttacaggacagcagatggggaattcatggctgttggagcaatagaaccccagttctacgagctgctgatcaaaggctgctgggggatgcttcagtttccttcacagaaaactaaatag

SEQ ID NO: 17 - DNA, 1185 bp, Homo sapiens
atggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtgttctctcaaaaattggcagaagtggtgagaatccgtatgccccgctgaatctcctggctgactttgctggtggtggccttatgtgtgcactgggcattataatggctctttttgaccgcacacgcactggcaagggtcaggtcattgatgcaaatatggtggaaggaacagcatatttaagttcttttctgtggaaaactcagaaatcgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctgatgaacttcccaatcagatgagcatggatgattggccagaaatgaagaagaagtttgcagatgtatttgcaaagaagacgaaggcagagtggtgtcaaatctttgacggcacagatgcctgtgtgactccggttctgacttttgaggaggttgttcatcatgatcacaacaaggaacggggctcgtttatcaccagtgaggagcaggacgtgagcccccgccctgcacctctgctgttaaacaccccagccatcccttctttcaaaagggatcctttcataggagaacacactgaggagatacttgaagaatttggattcagccgcgaagagatttatcagcttaactcagataaaatcattgaaagtaataaggctggtagcaagttctggatcttatacccaacacacagcaacatccagaaataa